(19) United States
(12) Patent Application Publication
(10) Pub. No.: US 2009/0088411 A1
(43) Pub. Date: Apr. 2, 2009

(54) No-donating corticosteroid with improved pharmacokinetic, anti-inflammatory and vasodilatory properties

(75) Inventors: Paolo Renzi, Westmount (CA); Rene Pageau, Kirkland (CA); Mark Parry-Billings, Westmount (CA)

Correspondence Address: Davidson, Davidson & Kappel, LLC, 485 7th Avenue, 14th Floor, New York, NY, 10018, US

(73) Assignee: Topigen Pharmaceuticals Inc., Montreal (CA)

(21) Appl. No.: 12/228,045

(22) Filed: Aug. 8, 2008

Related U.S. Application Data
(60) Provisional application No. 60/969,244, filed on Aug. 31, 2007.
(60) Provisional application No. 61/065,399, filed on Feb. 8, 2008.
(60) Provisional application No. 61/065,398, filed on Feb. 8, 2008.
(60) Provisional application No. 61/065,396, filed on Feb. 8, 2008.

(51) Int. Cl. A61K 31/58

(52) U.S. Cl. 514/174

(57) ABSTRACT

There are herein provided methods of treatment and nitric oxide donating compositions of matter for the treatment of respiratory diseases and associated conditions.

### CROSS-REFERENCE TO RELATED APPLICATIONS

This is a continuation-in-part of U.S. Provisional patent application Ser. No. 60/969,244 filed Aug. 31, 2007; Ser. No. 61/065,399, filed Feb. 8, 2008; Ser. No. 61/065,398, filed Feb. 8, 2008; and Ser. No. 61/065,396, filed Feb. 8, 2008; each of which are hereby incorporated by reference in their entireties.

### FIELD OF THE INVENTION

The present invention relates to the use of steroidal compounds having improved pharmacokinetic and pharmacological activities. In particular the invention relates to steroidal compounds, which when administered by inhalation, are having much less systemic distribution. This unique and unexpected pharmacokinetic effect is associated with improved pharmacological activities, in particular:

those affecting pro-inflammatory markers;

those affecting inflammatory cells;

those affecting blood pressure.

The invention also relates to methods to treat illnesses wherein steroidal compounds are generally applied, with increased benefit in terms of safety and broader efficacy

### BACKGROUND OF THE INVENTION

The alveolar and airway epithelium is recognized as a dynamic barrier that plays an important role in regulating inflammatory and metabolic responses to oxidative stress, sepsis, endotoxemia, and other critical illnesses in the lung. The respiratory epithelium, in particular, is a primary target of inflammatory conditions/infections at the epithelial-blood interface, and is itself capable of amplifying an inflammatory signal by recruiting inflammatory cells and producing inflammatory mediators.

Chronic Obstructive Pulmonary Disease (COPD) is one example of an inflammatory airway and alveolar disease where persistent upregulation of inflammation is thought to play a role. Inflammation in COPD is characterized by increased infiltration of neutrophils, CD8 positive lymphocytes, and macrophages into the airways. Neutrophils and macrophages play an important role in the pathogenesis of airway inflammation in COPD because of their ability to release a number of mediators including elastase, metalloproteases, and oxygen radicals that promote tissue inflammation and damage. It has been suggested that inflammatory cell accumulation in the airways of patients with COPD is driven by increased release of pro-inflammatory cytokines and of chemokines that attract the inflammatory cells into the airways, activate them and maintain their presence. The cells that are present also release enzymes (like metalloproteases) and oxygen radicals which have a negative effect on tissue and perpetuate the disease. A vast array of pro-inflammatory cytokines and chemokines have been shown to be increased within the lungs of patients with COPD. Among them, an important role is played by tumor necrosis factor alpha (TNF-alpha), granulocyte-macrophage colony stimulating factor (GM-CSF) and interleukin 8 (IL-8), which are increased in the airways of patients with COPD. Although primarily affecting the lung, COPD also has systemic effects; for example, C-reactive protein (CRP) is increased in COPD and has been associated with an increased risk of incident myocardial infarction, stroke, unstable angina, and sudden coronary death. Indeed, most of the deaths in mild to moderate COPD have been associated with cardiovascular events. Any therapy for COPD that would also positively affect factors associated with cardiovascular events would be of benefit in this disease. Inhaled corticosteroids are currently employed in patients with COPD but have been associated with an increased risk of pneumonia.

Bronchial asthma is widely recognized as a complex chronic inflammatory disorder of the airways characterized by acute episodes of airway obstruction and increased responsiveness and reduced respiratory function. In the course of an inflammatory response, many types of inflammatory cells such as macrophages, lymphocytes (especially Th2 lymphocytes), mast cells, eosinophils, basophils, neutrophils, and platelets are involved, all of which release inflammatory mediators in the airways. The inflammatory mediators implicated in asthma include: histamine, serotonin, adenosine, prostaglandins (PGs), leukotrienes, bradykinin, substance P, and platelet activating factor. Inflammatory mediators produce many effects in the airways, including bronchoconstriction, plasma exudate, mucous secretion, hyperresponsiveness, neural effects, and attraction and activation of inflammatory cells (Barnes et al., 1998). For certain types of asthma, neutrophils make significant contributions to the pathophysiology of the disease [e.g., patients with asthma who smoke, acute exacerbations of asthma, severe asthma that is neutrophil dependent (˜50% of patients).

In the past decade, the treatment of asthma has emphasized long-term suppression of airway inflammation (e.g., corticosteroids) plus relief of symptoms (e.g., β₂-adrenergic agonists) (Serafin, 1996). Inhaled corticosteroids are currently the first-line treatment for asthma in many countries. However, used alone or in combination with other therapies, corticosteroids do not consistently abrogate airway inflammation in patients with asthma. Moreover, their potential side effects, mainly osteoporosis, limit their use in escalating doses (Busse and Lemanske, 2001). Bone loss during corticosteroid treatment is mediated by inhibition of gonadal and adrenal steroid production, leading to hypogonadism and a direct negative effect on calcium absorption and osteoblast function (Canalis and Delany, 2002). Epidemiological data suggest that corticosteroid treatment doubles the risk of fractures of the hip and distal radius and at least quadruples the risk of vertebral fractures (Lips, 1999). It is thus important to employ the lowest dose of inhaled corticosteroids to maintain adequate control of asthma with the least amount of systemic distribution possible. The Canadian asthma consensus guidelines recommend that if asthma is not adequately controlled on a low dose of inhaled corticosteroids one should add an additional bronchodilator or an anti-inflammatory therapy (e.g., anti-leukotriene) (Lemiere, 2004). For neutrophil related asthma, it is optimal if a therapeutic agent possesses both bronchodilation and anti-neutrophil effects.

Other examples of respiratory diseases where inflammation seems to play a role include: eosinophilic cough, bronchitis, acute and chronic rejection of lung allograft, sarcoidosis, pulmonary fibrosis, rhinitis and sinusitis.

Eosinophilic cough is characterized by chronic cough and the presence of inflammatory cells, mostly eosinophils, within the airways of patients in the absence of airway obstruction or hyper responsiveness. Several cytokines and chemokines are increased in this disease, although they are mostly eosinophil directed. Eosinophils are recruited and activated within the airways and potentially release enzymes and oxygen radicals that play a role in the perpetuation of inflammation and cough.

Acute bronchitis is an acute disease that occurs during an infection or irritating event for example by pollution, dust, gas or chemicals, of the lower airways. Chronic bronchitis is defined by the presence of cough and phlegm production on most days for at least 3 months of the year, for 2 years. One can also find during acute or chronic bronchitis within the airways inflammatory cells, mostly neutrophils, with a broad array of chemokines and cytokines. These mediators are thought to play a role in the inflammation, symptoms and mucus production that occur during these diseases.

Lung transplantation is performed in patients with end stage lung disease. Acute and more importantly chronic allograft rejection occur when the inflammatory cells of our body, lymphocytes, do not recognize the donor organ as “self”. Inflammatory cells are recruited by chemokines and cytokines and release a vast array of enzymes that lead to tissue destruction and in the case of chronic rejection a disease called bronchiolitis obliterans.

Sarcoidosis is a disease of unknown cause where chronic non-caseating granulomas occur within tissue. The lung is the organ most commonly affected. Lung bronchoalveolar lavage shows an increase in mostly lymphocytes, macrophages and sometimes neutrophils and eosinophils. These cells are also recruited and activated by cytokines and chemokines and are thought to be involved in the pathogenesis of the disease.

Pulmonary fibrosis is a disease of lung tissue characterized by progressive and chronic fibrosis (scarring) which will lead to chronic respiratory insufficiency. Different types and causes of pulmonary fibrosis exist but all are characterized by inflammatory cell influx and persistence, activation and proliferation of fibroblasts with collagen deposition in lung tissue. These events seem related to the release of cytokines and chemokines within lung tissue.

Acute rhinitis is an acute disease that occurs during an infection or irritating event, for example, by pollution, dust, gas or chemicals, of the nose or upper airways. Chronic rhinitis is defined by the presence of a constant chronic runny nose, nasal congestion, sneezing and pruritis. One can also find within the upper airways during acute or chronic rhinitis inflammatory cells with a broad array of chemokines and cytokines. These mediators are thought to play a role in the inflammation, symptoms and mucus production that occur during these diseases.

Acute sinusitis is an acute, usually infectious disease of the sinuses characterized by nasal congestion, runny, purulent phlegm, headache or sinus pain, with or without fever. Chronic sinusitis is defined by the persistence for more than 6 months of the symptoms of acute sinusitis. One can also find during acute or chronic sinusitis within the upper airways and sinuses inflammatory cells with a broad array of chemokines and cytokines. These mediators are thought to play a role in the inflammation, symptoms and phlegm production that occur during these diseases.

There is a growing body of evidence suggesting an intimate link between inflammation and neoplastic diseases. The tumor microenvironment is shaped by cells entering it, and their functions reflect the local conditions. Successive changes occurring at the tumor site during tumor progression resemble chronic inflammation. This chronic inflammatory reaction seems to be largely orchestrated by the tumor, and it seems to promote tumor survival. It has become evident that early and persistent inflammatory responses observed in or around developing neoplasms regulates many aspects of tumor development (matrix remodeling, angiogenesis, malignant potential) by providing diverse mediators implicated in maintaining tissue homeostasis, e.g., soluble growth and survival factors, matrix remodeling enzymes, reactive oxygen species and other bioactive molecules.

As described above, these inflammatory respiratory diseases or diseases in which inflammation plays a critical role are all characterized by the presence of mediators that recruit and activate different inflammatory cells which release enzymes or oxygen radicals causing symptoms, the persistence of inflammation and when chronic, destruction or disruption of normal tissue.

A logical therapeutic approach would be to downregulate cytokine and chemokine production and the inflammatory cell response. This has been performed in all the diseases described above by employing either topical or systemic corticosteroids with different levels of success. Corticosteroids are immune suppressive and have effects not only on inflammatory cells but also on other cells of the body that could lead to toxicity when administered chronically. As mentioned previously inhaled corticosteroids do not have any significant anti-neutrophil properties. The need to have steroidal compounds with improved pharmacological efficacy and fewer side effects is obvious. The applicant has unexpectedly found that a specific class of steroidal compounds showed increased and selective anti-inflammatory efficacy toward inflammatory markers, and inflammatory cells; less systemic distribution and decreased systemic blood pressure. These characteristics should lead to a better therapy of inflammatory diseases with less potential for systemic toxicity and additional systemic effects improving cardiovascular prognosis.

### SUMMARY OF THE INVENTION

The present invention relates to the use of steroidal compounds having an improved pharmacological activity and lower side effects for inflammatory and more specifically inflammatory respiratory conditions.

In particular, the invention relates to the use of steroidal compounds that have been modified by the addition of a nitric oxide donating moiety which have an improved pharmacological activity and lower side effects, in particular:
those affecting the systemic distribution from the lung tissue;
those affecting pro-inflammatory markers;
those affecting inflammatory cells;
those affecting blood pressure.

The present invention provides the use of a steroidal structure which:

exhibits a longer residence time in the lung tissue (increased Tₘₐₓ); or

is less bio-available from the systemic circulation (reduced Cₘₐₓ and AUC);

The present invention further relates to the use of compounds having a steroidal structure having an improved anti-inflammatory activity in the lung tissue toward neutrophils, and at peripheral systemic levels of the C-reactive protein.

The present invention also relates to the use of compounds, which when delivered to the lung by inhalation, have systemic vaso-dilating activity as well as the following:

decreased systemic distribution of the parent steroid compound and its metabolite(s) in animals and humans with or without lung disease;

anti-inflammatory effects on neutrophils, the unexpected effect being the decrease in sputum if neutrophils are elevated but no effect if they are within the normal range;

a better effect on CRP, a marker of CV prognosis in CV and respiratory diseases; or

a decrease in systemic blood pressure, an effect that is clinically significant after inhalation when no effects on systemic blood pressure were reported in the inhaled NO study in patients with COPD, it being known that a decrease in blood pressure improve cardiovascular prognosis in patients at risk of the disease.

More particularly, the present invention is directed to a method for treating a respiratory disease in a patient comprising administering a therapeutically effective amount of a compound having the following general structure, Formula 3:

A-W 3

wherein A is a corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide; and

wherein W is any nitric oxide (“NO”) donating moiety attached thereto capable of donating, releasing and/or directly or indirectly transferring any of the three redox forms of nitrogen monoxide (NO⁺, NO⁻, NO.) that is linked to the steroid backbone by a linking structure that is optionally cleavable in vivo by hydrolase mediated hydrolysis, such that there is a statistically significant decrease in the blood pressure of the patient compared to the baseline blood pressure of the patient prior to the administration of the compound. By a statistically significant decrease in blood pressure it is meant that such decrease is considered clinically relevant.

In certain embodiments, the administration of the compounds contemplated for use in accordance with the present invention may provide a therapeutic reduction in blood pressure in patients suffering from high blood pressure. It will be appreciated that further embodiments of the invention contemplate a method of treatment wherein the additional step of identifying those patients having high blood pressure, and treating such patients in accordance with the invention. More particularly, W is selected from one of Formulae A-G as further described herein as well as in the appended claims.

Additionally, the present invention relates to lower than expected systemic levels of an inhaled steroid in patients clinically indicated for treatment of respiratory diseases using steroid therapy. The steroids as contemplated for use in the present invention provide for reduced systemic steroid levels while advantageously achieving a therapeutic respiratory effect with a reduction in undesirable systemic side effects such as HPA axis suppression, which may be indicated, by reduced free urinary cortisol levels. This results in a safer steroid with a potent therapeutic effect.

The present invention also provides for a method of decreasing a sputum neutrophil level in a patient comprising administering to the patient a therapeutically effective amount of a compound having Formula 3 such that there is a statistically significant decrease in the sputum neutrophil level of the patient. The term “statistically significant decrease” is intended herein to mean clinically relevant.

Additionally, the present invention provides for a method of decreasing a plasma CRP level in patients having a respiratory disease comprising administering to the patients a therapeutically effective amount of a compound having Formula 3 such that there is a statistically significant decrease in the plasma CRP level in the patients.

More particularly, the present invention also provides for the use of a compound having Formula 3 for decreasing a sputum neutrophil level in patients, wherein the sputum neutrophil level of the patients is greater than 20% as well medicaments incorporating such compounds for such use.

Another embodiment of the present invention provides for the use of a compound having Formula 3 for decreasing plasma CRP levels in patients as well medicaments incorporating such compounds for such use.

Another intended embodiment of the present invention provides for a method of treating patients having (a) a respiratory disease associated with inflammation and (b) high blood pressure, that comprises administering a therapeutically effective amount of a compound having Formula 3.

Similarly, the present invention also provides for a method of treating patients having (a) a respiratory disease associated with inflammation and (b) a sputum neutrophil level of greater than 20%, that comprises administering a therapeutically effective amount of a compound having Formula 3.

A yet further embodiment provides for a method of treating patients having (a) a respiratory disease associated with inflammation and (b) a high plasma CRP level, comprising administering a therapeutically effective amount of a compound having Formula 3.

Still further, the present invention provides for a unique use of a compound having Formula 3 for treating a respiratory disease associated with inflammation in patients having at least one of high blood pressure, a high plasma CRP level and a sputum neutrophil level of greater than 20% as well medicaments incorporating such compounds for such use.

A still further embodiment of the present invention provides for a method of treating a respiratory disease in humans, comprising administering to human patients a compound having Formula 3. In accordance with such embodiment, the compound is administered at an effective dosing interval and in an amount effective to provide a statistically significant reduction in systolic blood pressure when measured in the patients 15 days after initiating treatment with the compound.

The present invention also provides for a method for treating a respiratory disease in humans that comprises administering to human patients having a respiratory disease and a sputum neutrophil level greater than about 20% a compound having Formula 3. In accordance with such embodiment, the compound is administered at an effective dosing interval and in an amount effective to provide a statistically significant decrease in sputum neutrophil level in the patients when measured in the patients 22 days after initiating treatment with the compound.

Another intended embodiment of the present invention provides for a method for treating a respiratory disease in humans, comprising administering to human patients having a respiratory disease and a sputum neutrophil level greater than about 20% a compound having Formula 3. In accordance with such embodiment, the compound is administered at an effective dosing interval and in an amount effective to provide a statistically significant decrease in plasma CRP level in the patients when measured in the patients 22 days after initiating treatment with the compound.

A yet further embodiment provides for a method for treating patients having (a) a respiratory disease associated with inflammation and (b) high blood pressure, comprising administering a therapeutically effective amount of a compound having Formula 3.

A further method of the present invention provides for treating patients having (a) a respiratory disease associated with inflammation and (b) a sputum neutrophil level of greater than 20%, comprising the administration of a therapeutically effective amount of a compound having Formula 3.

A further embodiment of the present invention provides for method of treating patients having (a) a respiratory disease associated with inflammation and (b) a high plasma CRP level, comprising administering a therapeutically effective amount of a compound having Formula 3.

A still further embodiment of the present invention provides for a method of treating a respiratory disease in patients comprising administering to the patients a therapeutically effective amount of a compound having Formula 3 such that the free urinary cortisol levels remain substantially unchanged from the pre-treatment levels in the patients.

The present invention also provides for a method of maintaining patients' pre-treatment free urinary cortisol levels while the patients are being treated for a respiratory disease comprising administering to the patients a therapeutically effective amount of a compound having Formula 3.

Moreover, the present invention provides for the use of a compound having Formula 3 for maintaining free urinary cortisol levels in patients that are indicated for receiving steroid therapy for a respiratory disease as well as the use of such compounds in the preparation of a medicament for the same, all of the foregoing being more particularly described in the detailed description the scope of which is set forth in the appended claims.

### BRIEF DESCRIPTION OF THE DRAWINGS

FIG. 1 shows a graphic depiction of adverse events reported by patients receiving Budeosnide or TPI-1020.

FIG. 2 shows effects of budesonide or TPI-1020 on supine and standing blood pressure.

FIG. 3 shows effects of budesonide or TPI-1020 on urinary cortisol.

FIG. 4 shows effects of budesonide and TPI-1020 on sputum neutrophils.

FIG. 5 shows acute effects of budesonide and TPI-1020 on FEV.

FIG. 6 shows a pharmacokinetic plasma profile of budesonide.

FIG. 7 shows plasma levels of budesonide at Day 1 (Budesonide Group).

FIG. 8 shows plasma levels of budesonide at Day 14 (Budesonide Group).

FIG. 9 shows plasma levels of budesonide at Day 1 (TPI-1020 Group).

FIG. 10 shows plasma levels of budesonide at Day 14 (TPI-1020 Group).

FIG. 11 shows Free Cortisol Levels (ITT) in urine.

### DETAILED DESCRIPTION

The present invention is directed to the use of NO-donating steroidal compounds having an improved pharmacological activity and lower side effects for inflammatory and, more specifically, inflammatory respiratory conditions. In particular the invention relates to the use of these steroidal compounds that have an improved pharmacological activity and lower side effects.

By way of example, bronchial asthma is widely recognized as a complex chronic inflammatory disorder of the airways characterized by acute episodes of airway obstruction, increased airways responsiveness and occasionally reduced pulmonary function (ref). In this disease exhaled nitric oxide (eNO) is increased when compared to normal individuals and eNO decreases when asthma improves. A discrepancy exists between the association of this decrease in eNO when asthma improves and the potential benefits of NO on inflammation and broncho-constriction. Indeed, inhaled NO may be beneficial by reducing inflammatory cell adhesion and infiltration into the airways as well as by promoting bronchodilation. Hogman et al., demonstrated improved specific airway conductance after the inhalation of 80 parts per million (ppm) NO in nonsmoking subjects with hyperactive airways and in individuals with a clinical diagnosis of asthma who were being maintained on short-acting β2 agonists and inhaled corticosteroids. Whether administering NO to patients with asthma that are receiving inhaled corticosteroids over a longer period of time is beneficial or detrimental has not been studied.

Additionally, the present invention relates to lower than expected systemic levels of an inhaled steroid in patients clinically indicated for treatment of respiratory diseases using steroid therapy. Inhaled steroids are known to cause a number of systemic adverse effects. One adverse effect is the suppression of the function of the HPA (hypothalamic-pituitary-adrenal) axis. This suppression is linked to side effects such as growth suppression in children and reduction in bone density in adults. This undesirable HPA axis suppression is typically measured by a reduction in urinary cortisol excretion. The effect on the HPA axis is directly linked to the level of steroid in the systemic circulation whereby higher levels typically result in more suppression. Systemic steroid load is typically reflected in patient AUC data wherein higher AUC levels correlate with higher levels of systemic steroid. The steroids as contemplated for use in the present invention provide for reduced systemic steroid levels while advantageously achieving a therapeutic respiratory effect with a reduction in undesirable systemic side effects such as HPA axis suppression that may be indicated by reduced free urinary cortisol levels. This results in a safer steroid with a potent therapeutic effect.

In accordance with the present invention, the term NO-donating steroidal compounds refers to compounds that have a steroidal backbone with a nitric oxide (“NO”) donating moiety attached thereto. Under certain physiological conditions these compounds are able to donate, release and/or directly or indirectly transfer any of the three redox forms of nitrogen monoxide (NO⁺, NO⁻, NO.), such that: a) the biological activity of the nitrogen monoxide species is expressed at the intended site of action, and/or; 2) endogenous production of nitric oxide in vivo is stimulated, and/or; 3) endogenous levels of nitric oxide are elevated in vivo, all the foregoing being further described in U.S. Pat. Nos. 7,282,519 and 7,244,753 the entireties of which are hereby incorporated herein by reference thereto.

It will be appreciated by those skilled in the art that the terms nitrosylated compounds or moieties and nitrosated compounds or moieties refer to substitution with at least one NO or NO₂ group, respectively. The term “nitro” refers to the group NO₂ thus “nitrosated” refers to compounds that have been substituted therewith. The term “nitroso” refers to the group NO thus “nitrosylated” refers to compounds that have been substituted therewith. “Thionitrate” refers to —S—NO₂. “Thionitrite” and “nitrosothiol” refer to —S—NO. “Nitrile” and “cyano” refer to —CN. Generally, nitric oxide donating groups can be added through, for example but not limited thereby, one or more sites such as oxygen (hydroxyl condensation), sulfur (sulfhydryl condensation) and/or nitrogen.

Known methods for nitrosating and/or nitrosylating compounds are described in U.S. Pat. Nos. 5,380,758, 5,859,053, 5,703,073, 6,297,260, and 6,696,592 the disclosures of each of which are incorporated by reference herein in their entirety, as well as in WO 94/03421, WO 94/04484, WO 94/12463, WO 95/09831, WO 95/19952, WO 95/30641, WO 97/27749, WO 98/09948, WO 98/19672, WO 98/21193, WO 00/51988, WO 00/61604, WO 00/72838, WO 01/00563, WO 01/04082, WO 01/10814, WO 01/12584, WO 01/45703, WO 00/61541, WO 00/61537, WO 02/11707, WO 02/30866 and in Oae et al, Org. Prep. Proc. Int., 15(3):165-198 (1983). The methods of nitrosating and/or nitrosylating the compounds described in these references can be applied by one skilled in the art to produce any of the nitrosated and/or nitrosylated compounds described herein.

These compounds as used in accordance with the present invention (including their corresponding stereoisomers, salts, solvates, esters, hydrates, polymorphs, prodrugs, and analogues thereof) are NO-donating steroid derivatives derived from the corticosteriod class having the same general ring structure as cortisol, shown below.

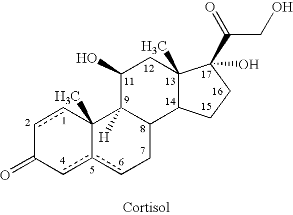

This class of compounds is exemplified by, but not limited to, the following steroids which may be used to form the corticosteroidal backbone of the compounds contemplated for use in accordance with the present invention: 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

It will be further appreciated that while the structures of the foregoing steroid family all approximate the ring structure of cortisol, various structural differences may occur from compound to compound with respect to the existing functional groups attached to the rings, especially at the C-11, C-16, and C-17 or C-21 positions, as illustrated by some of the well known compounds set forth below. Accordingly, the steroidal backbone to which the NO-donating moiety is attached may have multiple sites for attachment based upon the steroid selected as well as the availability of such sites present thereupon.

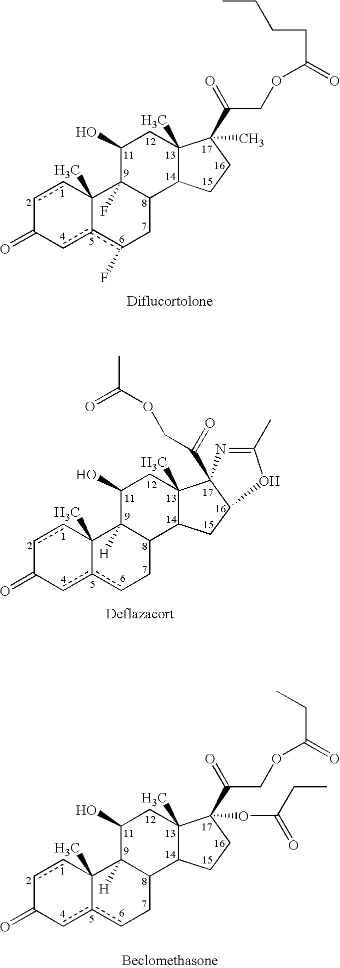

In accordance with the present invention, the steroid backbone is substituted with a NO-donating compound (see for example, 4′-nitrooxymethylbenzoic acid, shown below) through various well-known synthesis pathways readily familiar to those skilled in the art.

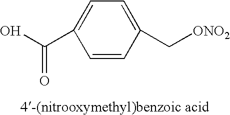

For example, U.S. Pat. No. 6,696,592, which is hereby incorporated herein by reference thereto, illustrates various pathways by which nitrooxyalkylbenzoic acid substituents can be attached to a steroid backbone at various hydroxyl sites present on a steroid, most preferably those hydroxyls positioned either directly or indirectly off the C-11 or the C-17 or C-21 carbons.

As previously mentioned, the site for attachment of the NO-donating moiety will of course depend upon the structure of the steroidal backbone that is selected as a recipient for such attachment. Further, it will be appreciated by the artisan that the site for attachment must also be chemically conducive to substitution with respect to such considerations as steric hindrances, bond formation constraints, synthesis limitations, and the like. Moreover, there may be simultaneous substitution with the same or a different NO-donating moiety off such multiple sites thereby rendering a multiple NO-donating effect for the resulting compound, providing however that the character of the sites as well as the moieties to be substituted therewith are chemically conducive to such a multiple substitution, the identification and appreciation of which will be readily apparent to one skilled in the art.

By way of further example, it will be readily apparent from the foregoing benzoic acid structure that the linkage must be facilitated through its carboxylic terminus, thereby rendering the NO-donating portion of the structure chemically unobstructed and available to perform a NO-donating function while the carboxylic terminus of the NO-donating moiety forms an ester linkage with the steroid.

Therefore, in one embodiment, the general structure of the NO-donating compounds as contemplated for use in accordance with the present invention is predicated upon the formation of an ester linkage between the parent steroid and the NO-donating moiety. Such a linkage can be directly adjacent to the ring structure as would be the case where the steroidal backbone has an existing hydroxyl group bonding directly to a carbon situated within the ring or, alternatively the linkage can be formed more distal to the ring structure. In either case, the linkage will be cleavable in vivo via hydrolase mediated hydrolysis, in this case esterase mediated hydrolysis, to render a dissociation of the parent steroid and the NO-donating moiety that is optimal for maximizing therapeutic effects of both portions of the molecule.

Examples of such cleavable linkers are exemplified by, but in no way limited to, esters, amides, carbamates, and carbonates that are cleavable via in vivo hydrolysis to yield a NO-donating moiety and the parent steroid. In one embodiment, as exemplified immediately below, the cleavable site is placed immediately distal to the C-21 hydroxyl group, however, the site of cleavage can be located more distal to the steroid backbone than the C-21 position provided that the portion of the moiety remaining attached thereto subsequent to cleavage does not impede either the steroid's or the NO-donating group's therapeutic functionality.

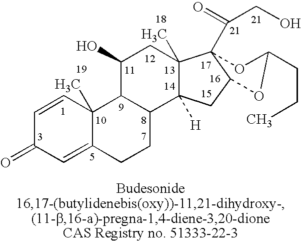

As mentioned in the case of 4′-nitrooxymethylbenzoic acid set forth above, the NO-donating moiety is linked to the steroid by way of its terminal carboxyl group which is available to undergo an esterfication reaction with any of several available functional groups, preferably hydroxyls that might be present on the parent steroid backbone. It will readily understood that these groups, the identification and availability of which will be immediately apparent to the artisan, must have the appropriate steric and chemical bonding character necessary for stable ester bond formation.

For example, in addition to U.S. Pat. No. 6,696,592 mentioned above, U.S. Pat. Nos. 5,837,698, 5,792,758 and 5,985,862 (all three of which are hereby incorporated herein by reference thereto) describe multiple hydroxyl attachment sites and synthesis methods that can be adapted for constructing compounds made in accordance with the present invention.

In yet another embodiment, the molecule is not cleaved in vivo but rather stays intact thus rendering the cleavable site optional. In such embodiments, the cleavable portion of the linking structure (an ester, amide, or carbamate linkage, for example) is either non-existent or otherwise chemically protected. In the case of the foregoing, it will be appreciated by those skilled in the art that any such linking structure must be compatible with the therapeutic functionality of both the NO-donating group as well as the parent steroid compound. Appropriate considerations regarding the same would include but are not limited to issues such as overall size, molecular weight, solubility, steric hindrances, and the like.

In light of the foregoing, the present invention is more particularly directed to a method for treating a respiratory disease in a patient comprising administering a therapeutically effective amount of at least one of the compounds set forth herein wherein the patient undergoes a reduction in blood pressure meaning that there is a statistically significant decrease in the blood pressure of the patient compared to the baseline blood pressure of the patient prior to the administration of the compound. By a statistically significant decrease in blood pressure it is meant that such decrease is considered clinically relevant.

In certain embodiments, the administration of the compounds contemplated for use in accordance with the present invention may provide a therapeutic reduction in blood pressure in patients suffering from high blood pressure. It will be appreciated that further embodiments of the invention contemplate a method of treatment wherein the additional step of identifying those patients having high blood pressure, and treating such patients in accordance with the invention.

Accordingly, the present invention is directed to a method for treating a respiratory disease in a patient comprising administering a therapeutically effective amount of a compound having the following general structure, Formula 3:

A-W 3

wherein A is a corticosteroid selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide; and

wherein W is any nitric oxide (“NO”) donating moiety linked to the steroid backbone by a linking structure that is optionally cleavable in vivo by hydrolase mediated hydrolysis and is capable of donating, releasing and/or directly or indirectly transferring any of the three redox forms of nitrogen monoxide (NO⁺, NO⁻, NO.) such that there is a statistically significant decrease in the blood pressure of the patient compared to the baseline blood pressure of the patient prior to the administration of the compound. By a statistically significant decrease in blood pressure it is meant that such decrease is considered clinically relevant.

A preferred embodiment of the present invention is directed to a composition and method for treating an animal host or patient comprising administering a bronchodilator in combination with a therapeutically effective amount of a compound A-W, above, wherein W is represented by Formula 3, as shown below:

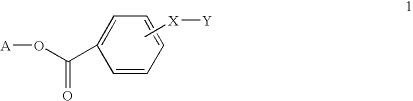

wherein A is the steroid or corticosteroid residue as described above,

X is a C₁-C₅ branched or linear chain alkyl; and

Y is either (ONO₂) or (ONO); with the proviso that A is linked to Formula 1B, directly or indirectly, the C-11 or the C-17 position of the steroid or corticosteroid residue, and preferably at the C-21 position of the steroid or corticosteroid residue when C-21 is present.

According to one aspect, the invention concerns use of a novel composition comprising a bronchodilator and a nitro-derivatized steroid, preferably a nitro-derivatized corticosteroid, of the following Formula 4:

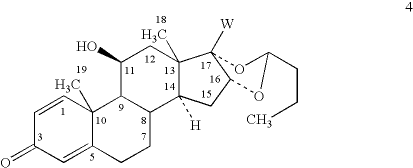

wherein W is any nitric oxide (“NO”) donating moiety attached thereto capable of donating, releasing and/or directly or indirectly transferring any of the three redox forms of nitrogen monoxide (NO⁺, NO⁻, NO.) for the treatment of respiratory disease associated with inflammation in a patient in need thereof. In certain preferred embodiments, W is a moiety as defined herein and is selected from one of the Formulae A-G described in the detailed description portion of this document as well as the appended claims.

Formula A compounds being represented by: —C(O)-L-(X_{O})—(X₁)—NO₂ (as described in published U.S. Patent Application Publication No. 2006/0052594 which is hereby incorporated herein by reference thereto) where L is defined as:

(CR₄R₅)ₙₐ(O)_{nb}(CR_{4′}R_{5′})ₙₐ(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;

X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and

X₁ is a bivalent-linking group selected from the following:

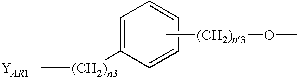

wherein n3 is an integer from 0 to 5 and n3′ is an integer from 1 to 3;

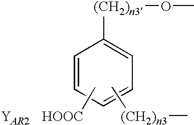

wherein n3 and n3′ have the above meaning or

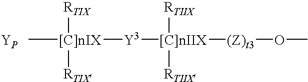

wherein:

nIX is an integer from 0 to 10, preferably 1-3;

nIIX is an integer from 1 to 10, preferably 1-5;

R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′}, are H;

Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;

t3 is zero or 1;

Z has the following meaning:

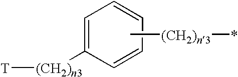

wherein:

* shows the position of the ONO₂ group;

T has the following meanings:

—COX₃—, —X₃CO—, wherein X₃═S or X_{O} as above defined;

—X₃— as above defined;

n3 and n′3 are as above defined.

In one preferred embodiment, Y³ is selected from the following bivalent radicals:

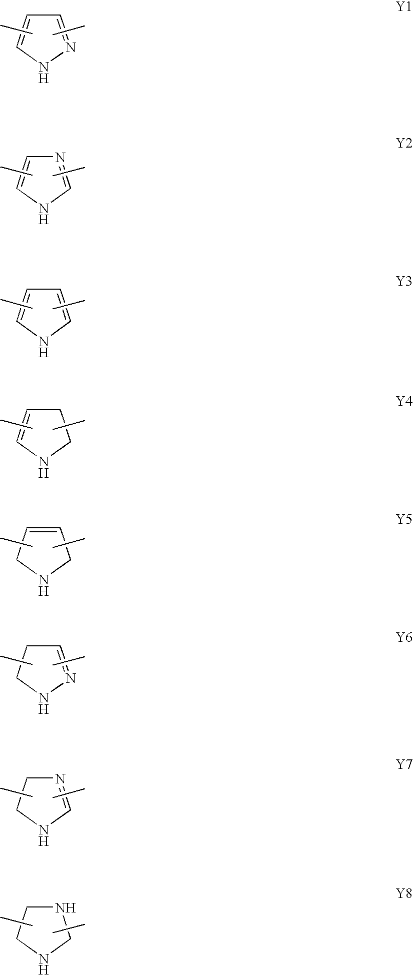

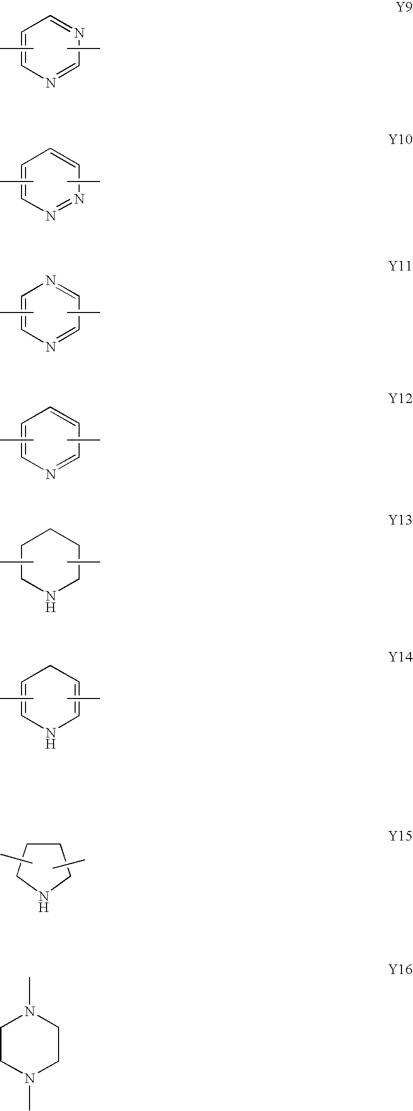

The following are preferred embodiments of Y³: (Y12), having the two free valences in the ortho positions with respect to the nitrogen atom; (Y16) with the two valences linked to the two heteroatoms, (Y1) (pyrazol) 3,5-disubstituted; (Y16) is also preferred.

In one preferred embodiment, L is na=n′b=1, n′a=2, n″b=n′″b=n″a=nb=0, R₄═CH₃, R₅═R_{4″}═R_{5″}═H. The precursors of the bivalent radicals X₁ (as above defined wherein the oxygen free valence is saturated with H and the free valence of the end carbon atom is saturated either with a carboxylic or hydroxyl or aminic group) are commercial products or they can be synthesized in accordance with known methods of the prior art.

The aforementioned compounds having a structure in accordance with Formula A of the present invention can be readily prepared with materials and methods well-known in the art, and particularly as disclosed in published U.S. Patent Application Publication No. 2006/0052594 the entirety of which is hereby incorporated herein by reference thereto.

More particularly, a first variation of the compounds of Formula A can be represented by:

—(CO-L)ₜ-(X)ₜ₁—X₁—NO₂

(as described in U.S. Pat. No. 6,610,676 which is hereby incorporated herein by reference) where t and t1 are integers=1 and L and X are defined as above as L and X₀, but where

X₁ is a bivalent-connecting bridge is selected from the group consisting of Y—O and Y_{1,}, wherein:

for Y—O, Y is a linear or whenever possible branched C₁-C₂₀ alkylene, preferably having from 2 to 5 carbon atoms, or an optionally substituted cycloalkylene having from 5 to 7 carbon atoms; and

for Y_{1,} Y is selected from Y_{AR1}, Y^{AR2}, and Y_{P}, as defined above, and is more particularly:

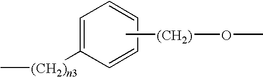

where n3 is an integer from 0 to 3;

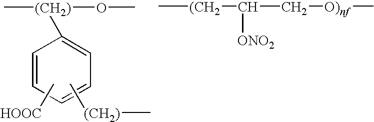

where nf′ is an integer from 1 to 6, preferably from 2 to 4; or

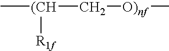

where R_{1f}═H, CH₃ and nf is an integer from 1 to 6, preferably from 2 to 4.

The aforementioned compounds of this first variation can be readily prepared and used with materials and methods well-known in the art, and particularly as disclosed in issued U.S. Pat. No. 6,610,676, the entirety of which is hereby incorporated herein by reference.

A second variation of the compounds of Formula A can be represented as:

—(CO-L)ₜ-(X)ₜ₁—X₁—NO₂

(as described in U.S. Pat. Nos. 7,056,905, 7,205,288, 7,196,075, 7,160,871, and 7,157,450 all of which being hereby incorporated herein by reference) where t and t1 are integers=1 and L is defined as above;

wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;

X is equal to O, C═O, NH, NR_{1c} where R_{1c} is a C₁-C₁₀, and preferably a C₁-C₄ linear or branched alkyl; OH, CH₃, Cl, N(—CH₂—CH₃)₂, SCH₂F, SH,

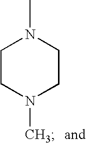

X₁ is a bivalent-connecting bridge is selected from the group consisting of Y—O and Y₁ as defined above.

The aforementioned compounds having a structure in accordance with this second variation of Formula A of the present invention can be readily prepared and used with materials and methods well-known in the art, and particularly as disclosed in issued U.S. Pat. Nos. 7,056,905, 7,205,288, 7,196,075, 7,160,871, and 7,157,450, and U.S. Patent Application Publication No. 2007/0238882, which are hereby incorporated herein by reference in their entirety.

A most preferred embodiment of a compound of Formula A is represented as

C(O)-L-(X₀)—(X₁)—NO₂

wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)

X₀ is C═O, and

X₁ is the bivalent-linking group Y_{AR1}, preferably

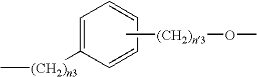

Formula B compounds being represented by: —C(O)CH₂O—X_{Z} (as described in U.S. Pat. No. 7,297,808 which is hereby incorporated herein by reference thereto) or alternatively by —C(O)CH₂OC(O)—X_{Z} wherein X_{Z} is defined as follows:

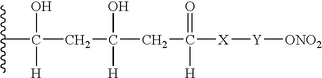

and X is O, S, NH or NHR₁, where R₁ is a straight or branched alkyl with 1 to 10 carbon atoms, preferably CH₃; and

Y is a bivalent radical having the following meanings a)-h)

a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃;

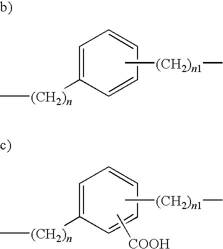

wherein for b) and c) above n is an integer from 0 to 20, and n¹ is an integer from 0 to 20;

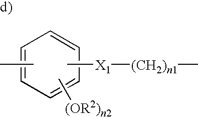

wherein, n¹ is as defined above and n² is an integer from 0 to 2; X₁ is —OCO— or —OCO— and R₂ is H or CH₃;

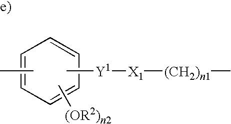

wherein n¹, n², R₂ and X₁ are as defined above; and Y¹ is either —CH₂—CH₂— or —CH₂═CH₂—(CH₂)ₙ₂—;

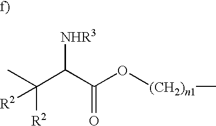

wherein n¹ and R² are as defined above; R³ is H or COCH₃; with the proviso that when Y is selected from the bivalent radicals mentioned under b) through f), the, —ONO₂ group is bound to —(CH₂)ₙ₁;

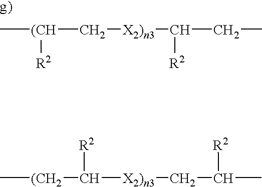

wherein X₂ is O or S, n³ is an integer from 1 to 6, preferably from 1 to 4, and R² is defined above;

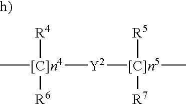

wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵, R⁶, and R⁷ are H; wherein the —ONO₂ group is bound to the following structure:

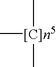

wherein, n⁵ is defined above;

Y² is a heterocyclic saturated, unsaturated or aromatic 5 or 6 members ring, containing one or more heteroatoms selected from nitrogen, oxygen, sulfur, and is selected from the following structures H1 through H13:

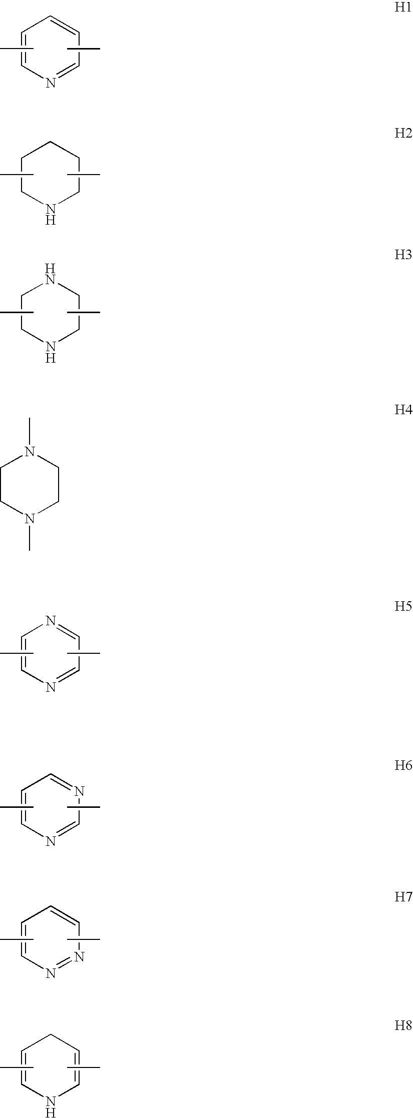

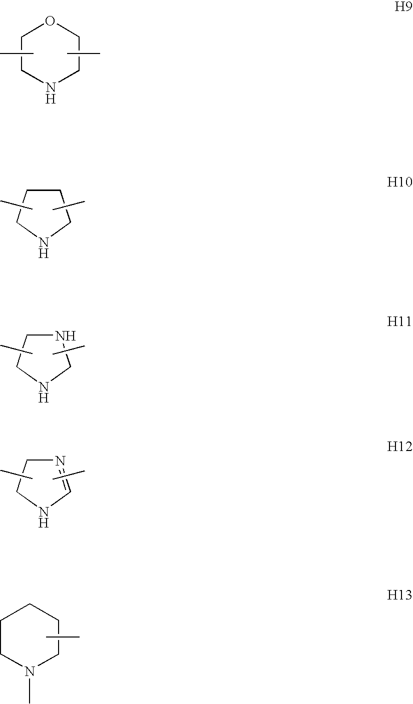

The aforementioned compounds having a structure in accordance with Formula B of the present invention can be readily prepared and used with materials and methods well-known in the art, and particularly as disclosed in issued U.S. Pat. No. 7,297,808 the entirety of which is hereby incorporated herein by reference thereto.

Formula C compounds being represented by: —C(O)CH₂O—(X₁—ONO₂)ₛ (as described in U.S. Pat. No. 7,217,733 which is hereby incorporated herein by reference thereto) or alternatively by —C(O)CH₂OC(O)—(X₁—ONO₂)ₛ wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—. The aforementioned compounds having a structure in accordance with Formula C of the present invention can be readily prepared and used with materials and methods well-known in the art, and particularly as disclosed in the aforementioned patent the entirety of which is, as previously stated, hereby incorporated herein by reference thereto.

Formula D compounds being represented by: —C(O)CH₂O—B—C) (as described in U.S. Pat. No. 7,199,258 which is hereby incorporated herein by reference thereto) wherein X₁ is defined as follows:

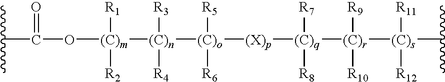

where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above.

The aforementioned compounds having a structure in accordance with Formula D of the present invention can be readily prepared and used with materials and methods well-known in the art, and particularly as disclosed in issued U.S. Pat. No. 7,199,258 the entirety of which is hereby incorporated herein by reference thereto.

Formula E compounds being represented by: —C(O)CH₂O—B—C^{Z}, various compounds of which are disclosed and/or exemplified in U.S. Pat. No. 5,837,698 as well as U.S. Pat. Nos. 5,792,758 and 5,985,862 (all three of which are hereby incorporated herein by reference thereto) C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO-donating portion of the compound, C^{Z}, via an amide, ester, carbamate or carbonate linkage that is induced adjacent to the 21 position.

In accordance with the various embodiments of the present invention, one embodiment provides using compounds of Formula H wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO-donating derivatives of the furoxans.

Alternatively, R₁ can be selected from any of the following chemical moieties that have been appropriately substituted with a NO-donating group: lower alkyls/alkenyls/alkynyls; that are substituted or unsubstituted (excepting out the necessary substitution with the NO-donating group as required in accordance with the present invention, the proviso being applicable in all the remaining groups recited below in this paragraph); substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.

In yet another embodiment, B—C^{Z} is equal to the following structure:

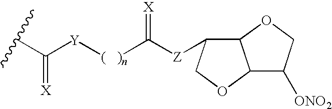

wherein, n is an integer from 1 to 4; X═O or S; Y=methylene, O, or NH₂; and Z=O or NH₂.

In another embodiment, the cleavable site is located more distal to the steroid backbone than in Formula E, provided that the portion of the moiety that remains attached to the steroid subsequent to cleavage does not impede either the steriod's or the NO-donating group's therapeutic functionality. In accordance with such an embodiment, W=Formula E′ where E′ is —C(O)CH₂—(B′—C^{Z}) wherein B′ is a spacer preferably containing 12 carbon atoms or less that connects the steroid backbone at the hydroxy immediately adjacent to the C-21 position with the NO-donating portion of the compound, C^{Z}, via an amide, ester, carbamate or carbonate linkage that is not adjacent to the C-21 position.

In accordance with the various embodiments of the present invention, one embodiment provides using compounds of Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO-donating derivatives of the furoxans.

Alternatively, R₁ can be selected from any of the following chemical moieties that have been appropriately substituted with a NO-donating group: lower alkyls/alkenyls/alkynyls; that are substituted or unsubstituted (excepting out the necessary substitution with the NO-donating group as required in accordance with the present invention, the proviso being applicable in all the remaining groups recited below in this paragraph); substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.

In accordance with the various embodiments of the present invention, another further embodiment provides using compounds of Formula E or E′ with the alternative structures readily discerned from U.S. Pat. Nos. 5,837,698, 5,792,758 and 5,985,862 the entireties of which are hereby incorporated herein by reference thereto. The aforementioned compounds having structures in accordance with Formula E or E′ of the present invention can be readily prepared with materials and methods well-known in the art, and particularly as disclosed in the aforementioned patents.

Formula F compounds being represented by: —C(O)CH₂O—K (as described in U.S. Pat. No. 7,282,519 which is hereby incorporated herein by reference thereto) or alternatively by —C(O)CH₂OC(O)—K wherein K is defined as follows:

—Y—(CR₄R_{4′})ₚ-T-(CR₄R_{4′})ₚ—ONO₂;

—Y—(CR₄R_{4′})ₒ-[phenyl]-T-(CR₄R_{4′})ₚ—ONO₂;

wherein T is ortho, meta or para;

—Y—B-[piperazinyl]-W—(CR₄R_{4′})ₚ—ONO₂;

—Y—(CR₄R_{4′})ₚ—V—B-T-(CR₄R_{4′})ₚ—ONO₂;

—Y—(CR₄R_{4′})ₚ-T-C(O)—(CR₄R_{4′})ₚ—(CH₂)—ONO₂;

—Y—(CR₄R_{4′})ₚ—C(Z)-(CH₂)_{q}-T-(CR₄R_{4′})_{q}—(CH₂)—ONO₂;

—Y—(CR₄R_{4′})-T-(CH₂)_{q}—V—(CR₄R_{4′})_{q}—(CH₂)—ONO₂;

—Y—(CR₄R_{4′})ₚ—V—(CH₂)_{q}—V—(CR₄R_{4′})_{q}—(CH₂)—ONO₂;

—Y—(CR₄R_{4′})ₒ—(W)_{q}—(CH₂)_{q}—V—(CR₄R_{4′})ₒ—(CH₂)—ONO₂;

—NRⱼ—O—(CH₂)ₒ—V—(CR₄R_{4′})_{q}—(CH₂)—ONO₂;

—NRⱼ—O—(CH₂)ₒ—(W)_{q}—(CR₄R_{4′})_{q}—(CH₂)—ONO₂;

—O—NRⱼ—(CH₂)ₒ—(W)_{q}—(CR₄R_{4′})_{q}—(CH₂)—ONO₂;

In accordance with the various embodiments of the present invention, another further embodiment provides using compounds of Formula F with the alternative structures readily discerned from U.S. Pat. No. 7,282,519 which is, as previously stated, hereby incorporated herein by reference thereto.

The aforementioned compounds having structures in accordance with Formula F of the present invention can be readily prepared with materials and methods well-known in the art, and particularly as disclosed in the aforementioned patent.

Formula G compounds being represented by: —C(O)CH₂O—X1 (as described in U.S. Pat. Nos. 7,256,205 and 7,244,753 which are both hereby incorporated herein by reference thereto) or alternatively by —C(O)CH₂OC(O)—X1 wherein X1 is defined as follows:

—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;

or alternatively, X1 is equal to K which is further defined as:

—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);

where, —(U)—(V) is equal to —Wᵢ-Eⱼ-W_{g}-[C—(Rₑ)(R_{f})]_{z}-T-Q;

or alternatively U and V are taken independently wherein U is O, S, or —N(Rₐ)(Rᵢ) and V is nitro, nitroso, or hydrogen;

wherein, a, b, c, d, g, i and j are each independently an integer from 0 to 3;

p, x, y and z are each independently an integer from 0 to 10;

Wₐ, W_{d}, Wᵢ, and W_{g} are independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;

E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;

h is an integer form 1 to 10;

q is an integer of from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;

k is an integer from 1 to 3;

T at each occurrence is independently a covalent bond, a carbonyl, an oxygen, —S(O)ₒ— or —N(Rₐ)(Rᵢ)—;

O is an integer from 0 to 2;

Rₐ is a lone pair of electrons, a hydrogen or an alkyl group;

Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ— T-Q, then the “-T-Q” subgroup designated as X can be a hydrogen, an alkyl, an alkoxy, an alkoxyalkyl, an aminoalkyl, a hydroxy, a heterocyclic ring or an aryl group.

In cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and [C—(Rₑ)(R_{f})]₂ denotes —C(Rₑ)(R_{f})—C(Rₑ)(R_{f})—.

In accordance with the various embodiments of the present invention, another further embodiment provides using compounds of Formula G with the alternative structures readily discerned from U.S. Pat. Nos. 7,256,205 and 7,244,753 which are, as previously stated, hereby incorporated herein by reference thereto.

The aforementioned compounds having structures in accordance with Formula G of the present invention can be readily prepared with materials and methods well-known in the art, and particularly as disclosed in the aforementioned patents.

It will be appreciated by those skilled in the art that further alternative embodiments of the foregoing compounds as contemplated for use in the present invention may be readily prepared using the various NO-donating groups set forth in U.S. Pat. Nos. 7,238,814, 7,235,237, 7,186,753, 7,186,708, 7,160,920, 7,122,539, 7,087,588, 6,987,120, 6,909,007, 6,869,974, 6,828,342, 6,794,372, 6,469,065, 6,645,965, 6,635,273, 6,613,784, 6,593,347, 6,579,863, 6,465,463, 6,433,182 6,417,207, 6,197,762, 6,143,734, 6,043,232, 5,780,495, 5,621,000, 5,189,034, RE37,116 as well as U.S. Patent Application Pub. Nos. 20070248676, 20070238740, 20070197499, 20070112194, 20070072854, 20070060586, 20070010571, 20060154905, 2006000943, 20050176694, 20040082652, and 200462243.

While it may be possible for the compounds as defined above to be administered as raw chemicals, it is preferable to use them in accordance with the present invention as a pharmaceutical formulation. Accordingly, the present invention provides a for the use of these compounds in a corresponding pharmaceutical formulation that comprises a one or more of the compounds as defined above invention (including their corresponding pharmaceutically acceptable stereoisomers, salts, solvates, esters, hydrates, polymorphs, prodrugs, and analogues thereof) together with one or more pharmaceutically acceptable carriers and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), and topical (including dermal, buccal, and sublingual) administration although the most suitable route may depend upon for example the condition and disorder of the recipient. The formulations may conveniently be presented in unit dosage form and may be prepared by any of the methods well known in the art of pharmacy. All methods include the step of bringing into association a preparation or a compound as defined above or a pharmaceutically acceptable salt or solvate thereof (“active ingredient”) with the carrier that constitutes one or more accessory ingredients. In general, the formulations are prepared by uniformly and intimately bringing into association the active ingredient with liquid carriers or finely divided solid carriers, or both, and then, if necessary, shaping the product into the desired formulation.

Formulations as contemplated for use with the present invention that are suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain antioxidants, buffers, bacteriostats and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Formulations for administration by inhalation can be prepared for use as aerosolized medicaments such as in manner recited in U.S. Pat. No. 5,458,135 and U.S. Pat. No. 5,447,150 which are hereby incorporated herein by reference thereto.

Preferred unit dosage formulations are those containing an effective dose, as hereinbelow recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds as contemplated for use in accordance with the present invention may be administered generally from about 100 ug to as much as 1 gram per day depending upon the method of administration as well as other clinical factors well known to those skilled in the art. For example, dosages for oral administration can range from about 100 ug to about 250 mg per day while dosages administered via intravenous methods may range from about 100 ug to about 1 g per day however the preferable route of administration is via inhalation where the dosages range from about 100 ug to about 2500 ug per day.

The compounds as used with in conjunction with the present invention are preferably administered by inhalation, orally or by injection (intravenous or subcutaneous). The precise amount of compound administered to a patient will be the responsibility of the attendant physician. However, the dose employed can depend on a number of factors, including the age and sex of the patient, the precise disorder being treated, and its severity. Also, the route of administration may vary depending on the condition and its severity.

Non-clinical pharmacological studies have provided evidence that preferred compounds of the present invention, NO-donating budesonide derivatives, may be effective in treating COPD and other respiratory diseases in humans where neutrophils are involved in the pathophysiology of the disease. However, it is highly likely that NO-donating budesonide derivatives will be employed in patients with asthma. Since an increase in eNO is associated with worsening asthma, it is important to show that these compounds remain safe and effective in patients with asthma, prior to evaluation of the compound in neutrophilic airways disease.

### EXAMPLE

### Clinical Study in Humans

A clinical study was undertaken in humans to test certain aspects of a preferred NO-donating budesonide derivative, TPI-1020, as compared to the corticosteroid, budesonide which was not modified with a NO-donating moiety. Specifically, the objectives of this study were to determine the safety, pharmacodynamic and pharmacokinetic activity of multiple doses of inhaled NO-donating budesonide derivatives compared to that of equimolar doses of inhaled budesonide in subjects with mild asthma who smoke. We chose patients with asthma who smoke since these patients had reported to have not only an increase in eNO but also an increase in neutrophils within their sputum (as described in COPD). While assessing the safety of these compounds in such patients we could thus explore the effects of the same in patients with increased sputum neutrophils.

Although this study was only powered for neutrophils, the following parameters were also evaluated: sputum total cell and differential cell counts, using known methods, on days 0, 15 and day 22; pre-dose FEV₁ measurements on days 0, 1, 14, 15 and 22; peak flow measurements throughout the study (miniwright meter); use of daily rescue medication (salbutamol) throughout the study; the response to methacholine challenge test; and the eNO levels (chemiluminescence after reaction with ozone) on days 0, 15 and 22 and plasma CRP levels on days 0, 15 and 22.

Pharmacodynamic effects on FEV₁ over 8 hours on days 1 and 14 were evaluated in a sub-group of 8-10 patients per group.

Safety parameters consisted of adverse events and serious adverse events, vital signs, asthma symptoms, laboratory determinations of CBC, urinalysis, regular blood biochemistry, plasma C-reactive protein (CRP), plasma and urinary free cortisol levels, spirometry and electrocardiograms (ECGs). The asthma control scoring system questionnaire (ACSS) was also used as a safety parameter. The ACSS questionnaire is made of three sections; a clinical score, the physiological score and the inflammation score. It was decided that for the physiological section of the questionnaire, the more informative FEV₁ would be preferred over the PEF or the diurnal fluctuation, for assessment. The plasma pharmacokinetic profile of budesonide was also determined on days 1 and 14 for up to 8 hours after a dose in a sub-group of 8-10 patients per group.

The methods of statistical analysis compared both arms by means of Students t-test, Chi² test, or Fisher exact test, as appropriate for each variable. All tests of hypotheses were two-sided with statistical significance defined as p≦0.05. Tests of normality were conducted for continuous variables, and as a result, the Wilcoxan rank sum test was used. Data are presented as mean±standard error of the mean (SEM) unless otherwise noted. When data were not normally distributed, results are presented as medians.

Test Subjects:

Individuals between 18 and 65 years old with stable, mild asthma (by American Thoracic Society (ATS) criteria), with a smoking history of 5-25 pack/years and still continuing to smoke despite medical advice, were eligible for enrolment. Subjects had not taken inhaled corticosteroids for at least one month. FEV₁ must have been ≧75% of predicted (at least 4 hours following use of rescue medication salbutamol) and reversible (≧12% and at least 200 mL after inhalation of salbutamol 200-400 μg during screening or within the last 6 months) or positive methacholine challenge test (PC₂₀≦16 mg/mL) within the last 5 years or a spontaneous fluctuation of FEV₁≧20% within the last 5 years.

Subjects who met the inclusion/exclusion criteria entered a 14-day run-in period with only a β-agonist bronchodilator (salbutamol) to use as needed. Subjects entered the therapeutic part of the study if they had a total symptom assessment score of ≧2 and <6 on at least 3 days of the last 7 days of the run-in period or a PEF diurnal variation >15% and <30% on at least 3 days of the last 7 days of the run-in period and urine cotinine level ≧200 ng/mL at the screening visit (indicative of an active smoker).

Those subjects who completed the 14-day run-in period were assigned to receive either an inhaled version of a compound of the present invention (in this case, budesonide 21-(4′-nitrooxymethyl)benzoate, designated hereafter as “TPI-1020”) or budesonide over a 21-day period. The doses administered were based on equimolar amounts of budesonide and were as follows: budesonide 400 μg bid for 14 days then 800 μg bid for 7 days; TPI-1020: 600 μg bid for 14 days then 1200 μg bid for 7 days. Study treatments were delivered in lactose-based blends using the capsule-based Aeroliser dry powder inhaler. Budesonide capsules (Miflonide®, Novartis Pharmaceuticals) delivered 383 μg as compared to 423 μg of budesonide for TPI-1020 (equivalent to 9.5% more budesonide delivered to the body in the TPI-1020 group). However, the fine particle dose of budesonide was 104 μg for the budesonide capsules and only 78 μg for the TPI-1020 capsules (equivalent to 25% less budesonide delivered to the lung in the TPI-1020 group). No other asthma medication except rescue salbutamol was permitted during the study.

A total of 27 subjects were enrolled into the study: 13 in the group receiving treatment with TPI-1020 and 14 in the group receiving budesonide treatment. One subject in the budesonide group withdrew prior to receiving medication resulting in 13 subjects in each group in the intent-to-treat population. Eight subjects in the TPI-1020 and 10 subjects in the budesonide group underwent pharmacokinetic and pharmacodynamic evaluations. All 26 subjects completed the study. The demographics of the patients in the two groups were not different (Table 1).

TABLE 1
Subject demographics

| | Budesonide (SE) | TPI 1020 (SE) |
|---|---|---|
| Age | 34.7 (2.6) | 34 (3.1) |
| Sex (M/F) | 7/7 | 7/6 |
| FEV1 | 3.3 (0.8) | 3.0 (0.5) |
| FEV1 % pred. | 87.4 (11) | 83.2 (8.9) |
| PEF AM | 414.8 (25) | 365.1 (26) |
| Rescue free | 40.7 (11) | 39 (11) |
| days (%) | | |
| PC20 mg/ml | 1.4 (1.3) | 3.5 (5.7) |
| Pack/years | 12.3 (1.8) | 9.3 (1.5) |
| Cigarettes/day | 12.8 (1.3) | 12.4 (1.3) |
| Urine cotinine | 1663 (240) | 1059 (182) |
| mg/ml | | |

There was also no difference between groups in haematology, chemistry, urine and ECG. All subjects were current smokers with a smoking history that was insufficient to induce clinically significant COPD.

The sample size was chosen to provide information about the primary study objective (safety) and with the power to detect a 20% difference in the decrease in sputum neutrophils with TPI-1020, versus that occurring with budesonide. There are no published studies in mild smokers with asthma that have described the variability in sputum neutrophils. However, based on the COPD publication by Gamble et al., almost all patients with COPD have increased sputum neutrophils (>20%). We calculated that if 13 subjects per group completed the study, a difference of 20% in neutrophils (%) between groups could be detected with a power of 80%.

### Results:

Safety:

(i) Adverse Events

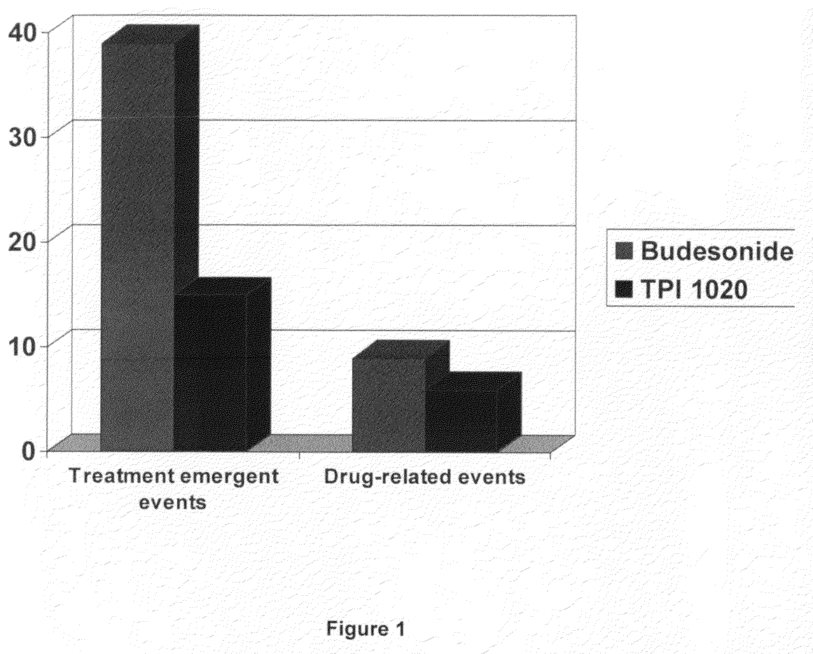

The total number of adverse events reported by patients receiving budesonide and TPI-1020 is graphically depicted in FIG. 1. More specifically, FIG. 1 depicts the total number of either treatment-related or physician-determined adverse events for each drug during the 21 day therapeutic period.

There were no serious adverse events during the study and only one severe adverse event during the study (headache in a budesonide-treated patient). There was no difference in the number of subjects that reported adverse events between TPI-1020 (n=6) and budesonide (n=7). However, there were three-fold less total treatment-emergent adverse events (n=13) in patients treated with TPI-1020 as compared to budesonide (n=39). There were also less drug-related adverse events in TPI-1020 treated patients than budesonide-treated patients (6 v 9).

(ii) Blood Pressure

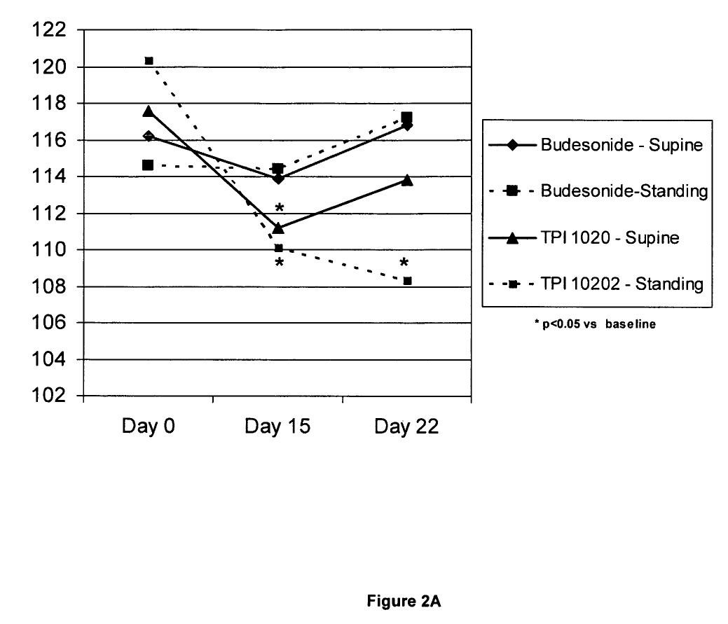

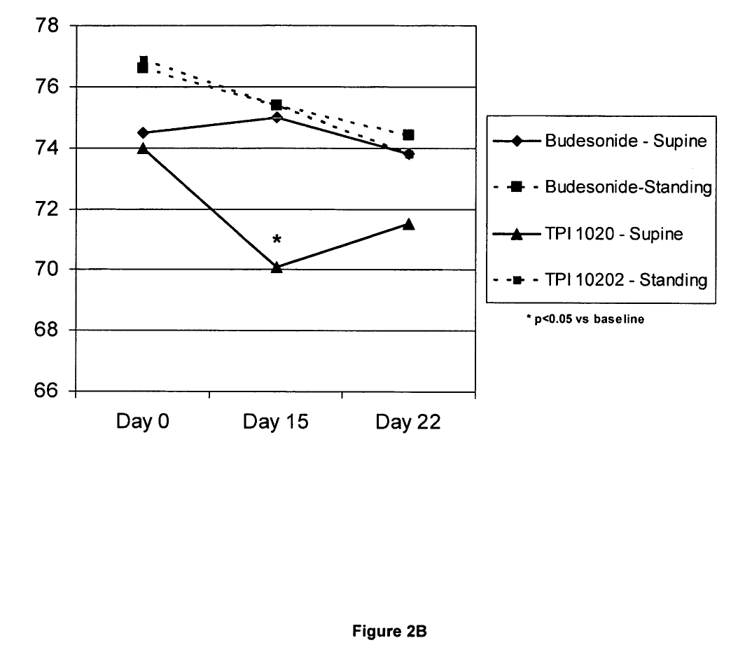

The effects of budesonide or TPI-1020 on blood pressure are presented in FIGS. 2a and 2b (p<0.05 vs baseline.) FIG. 2a shows standing and supine systolic blood pressure measurements, and FIG. 2b shows standing and supine diastolic blood pressure measurements. As shown, TPI-1020 decreased blood pressure values over the 22 day period but budesonide had no significant effects.

For supine diastolic, supine systolic, standing diastolic and standing systolic these decreases were 3.4, 3.2, 4.0 and 9.9%, respectively, at day 22 versus baseline. The differences were significantly different for at least one time point in supine diastolic, supine systolic and standing systolic. Standing pulse pressure also improved significantly after 22 days in the TPI-1020 group (−9.33 decrease in the TPI-1020 group versus +4.85 mmHg increase in the budesonide group, p=0.015). There were no orthostatic symptoms; the difference between supine and standing blood pressure on day 22 was not significantly different in TPI-1020-treated patients and clinically significant changes in systolic (>20 mmHg) and diastolic (>15 mmHg) blood pressure, were recorded seven times in three patients in each group.

(iii) Haematology Parameters

Treatment with neither medication had any effect on standard parameters of haematology, chemistry and urine except for blood urea nitrogen (BUN). BUN increased significantly in the budesonide-treated group from 4.2 (baseline) to 5.2 and 5.3 mmol/L on days 15 and 22, respectively (p<0.05 for both time points). BUN did not change in the TPI-1020-treated group (4.4, 3.9 and 4.4 mmol/L on days 0, 15 and 22, respectively). Neither treatments had any effect on the ECG, the QT and the QTc interval recorded in the pharmacodynamic group 20, 45 minutes, 3 hours and 8 hours post dose on day 1 after the first dose and on day 14.

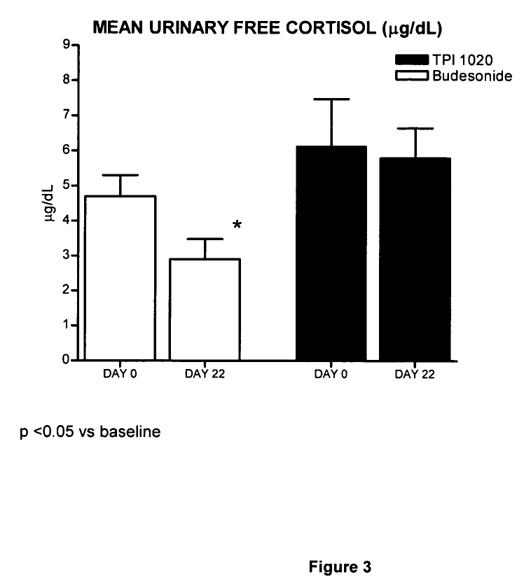

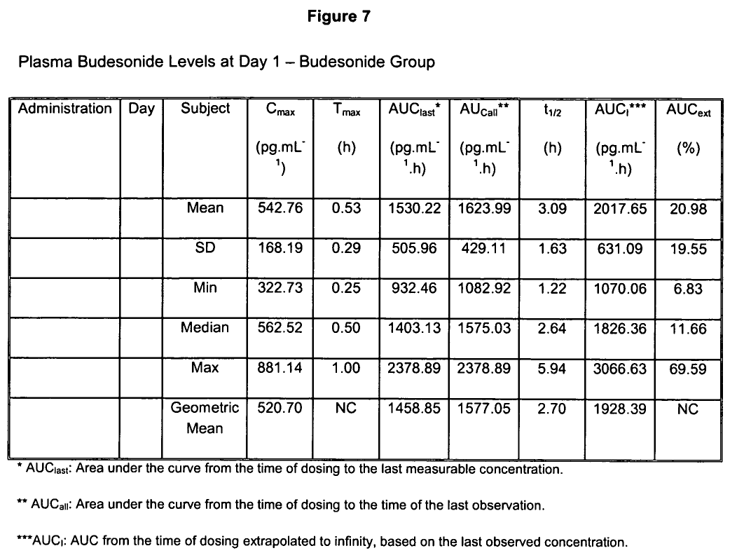

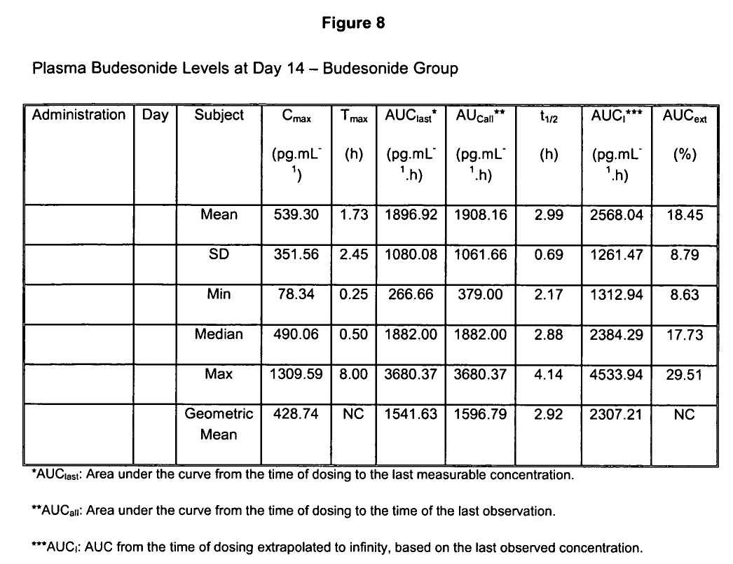

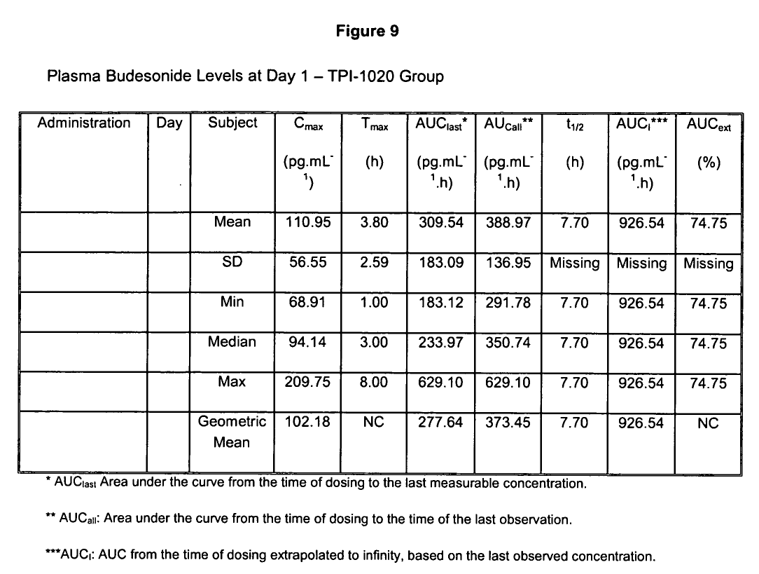

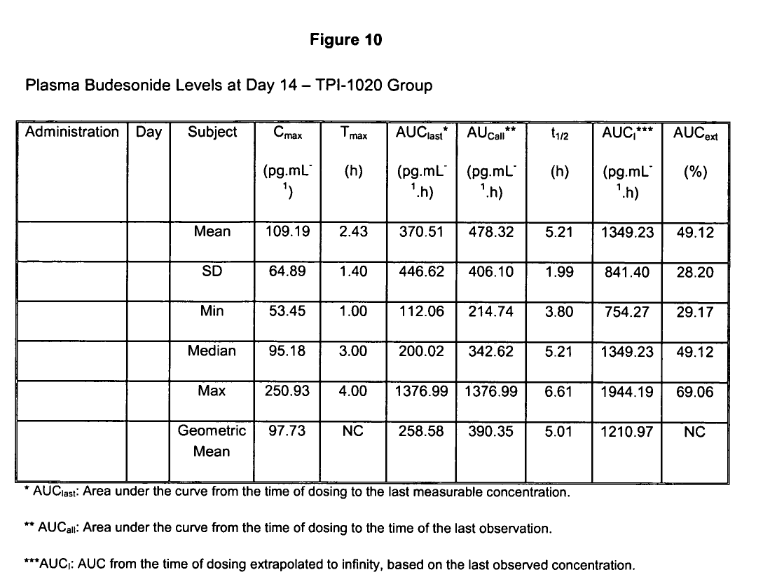

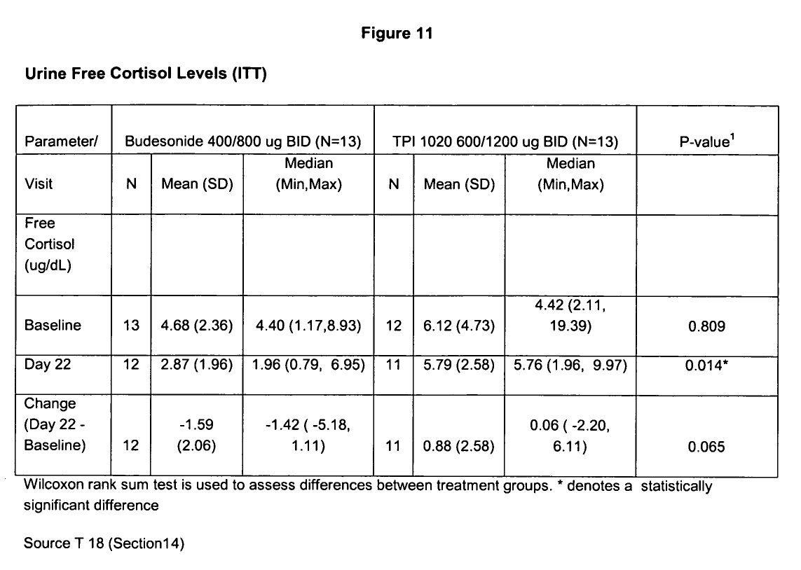

Both therapies also had no significant effects on morning plasma cortisol level (budesonide 13.1, 10.9 and 11.4 mcg/dl and TPI-1020 13.7, 14.5 and 11.7 mcg/dl at baseline, day 15 and day 22, respectively). However, budesonide significantly decreased 24-hour urinary free cortisol whereas TPI-1020 showed statistically insignificant effects relative to any decrease in urinary free cortisol as illustrated in FIG. 3, and FIGS. 7, 8, 9, 10, and 11. Specifically, FIG. 3 shows the effects of budesonide or TPI-1020 on 24 hour mean urinary cortisol. *p<0.05 vs baseline. FIG. 7 shows plasma budesonide levels at Day 1 for the subjects receiving budesonide. FIG. 8 shows plasma budesonide levels at Day 14 for the subjects receiving budesonide. FIG. 9 shows plasma budesonide levels at Day 1 for the subjects receiving TPI-1020. FIG. 10 shows plasma budesonide levels at Day 14 for the subjects receiving TPI-1020. FIG. 11 shows free cortisol levels (ITT) in using for all subjects.

Pharmacodynamic Activity:

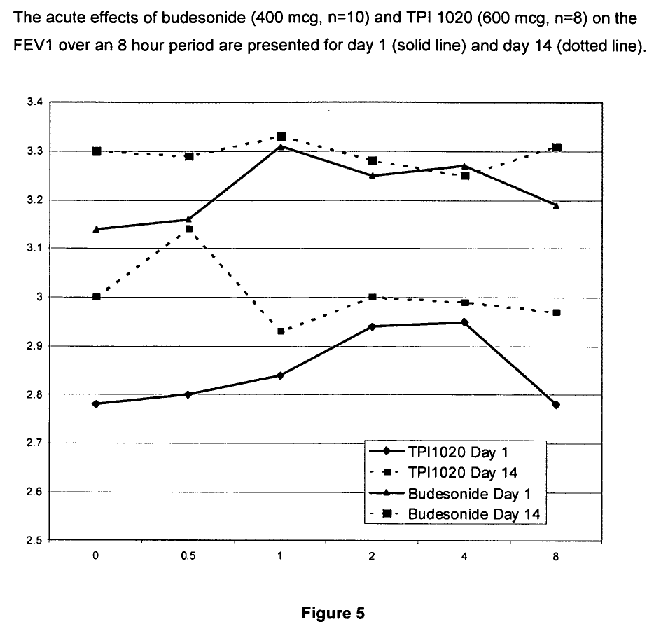

The effects of low dose TPI-1020 and of an equimolar dose of budesonide on FEV₁ over an 8 hour period on days 1 and 14 are presented in FIG. 5. FIG. 5 specifically shows acute effects of budesonide (400 mcg, n=10) and TPI-1020 (600 mcg, n=8) on the FEV1 over an 8 hour period are presented for day 1 (solid line) and day 14 (dotted line). TPI-1020, at the lowest dose studied, had no acute effects on FEV₁ when compared to budesonide on both days.

Pharmacokinetic Activity:

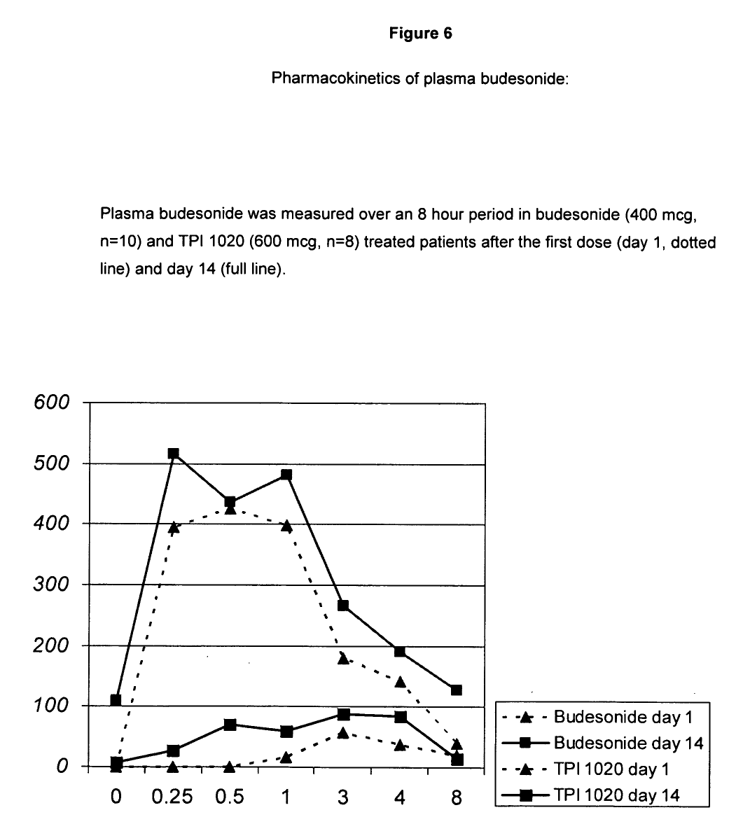

TPI-1020 was not detected in the plasma of treated subjects at all the time points that were assessed over an 8 hour period on days 1 and 14 and on day 22, approximately 12 hours after the last dose. The budesonide plasma phamacokinetic analysis was different in the TPI-1020 group when compared to the budesonide group (FIG. 6). The Cmax, Tmax, and AUC were, respectively in the TPI-1020 vs. the budesonide treated group. For the data presented in FIG. 6, plasma budesonide was measured over an 8 hour period in budesonide (400 mcg, n=10) and TPI-1020 (600 mcg, n=8) treated patients after the first dose (day 1, dotted line) and day 14 (solid line).

Effects on Sputum Neutrophils:

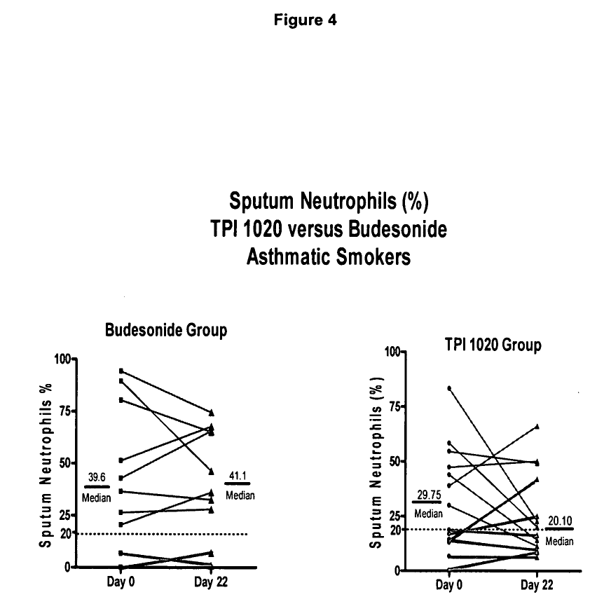

A trend was found for a decrease in sputum neutrophils in the TPI-1020 group. FIG. 4 shows the results of effects of budesonide and TPI-1020 on sputum neutrophils, presented as % sputum neutrophils on day 0 prior to therapy and day 22 (end of therapy). As shown in FIG. 4, edian sputum neutrophils changed from 39.6 to 41.1% in the budesonide group (3.7% increase) and from 29.8% to 20.1% in the TPI-1020 group (32.6% decrease). Since there was an unexpectedly large number of patients with normal baseline neutrophils in the sputum (9 of 26 patients with neutrophils <20%), a post hoc analysis was performed in patients with baseline sputum neutrophils >20% (abnormal) and those with sputum neutrophils <20% (normal). In those patients with high neutrophils at baseline, budesonide decreased sputum neutrophils from a mean of 55.1% to 51.9% over 22 days (5.8% decrease), decreased sputum neutrophils from 50.8% to 33.4% over 22 days (35.8% decrease). Whilst in those patients with normal neutrophils at baseline, neither budesonide nor TPI-1020 decreased sputum neutrophils (changes were from a mean of 4.25% to 3.95% for budesonide and from 11.5% to 17.9% over 22 days for TPI-1020). In addition, for patients with high neutrophils at baseline, TPI-1020 decreased sputum neutrophils to <30% in most (57.1%) patients, whilst for none of the patients treated with budesonide were neutrophils decreased to <30%.

Exploratory Assessment of Efficacy:

The effects of TPI-1020 and budesonide on physiology/symptoms and on inflammatory parameters are presented in Tables 2a and 2b, respectively.

TABLE 2a
Efficacy Physiology and Symptoms

| | Budesonide (SD) | TPI 1020 (SD) |
|---|---|---|
| FEV1 (ITT, L) | | |
| Baseline | 3.3 (0.8) | 3.0 (0.5) |
| Day 22 | 3.3 (0.8) | 3.0 (0.5) |
| FEV1 (PD, L) | | |
| Baseline | 3.2 (0.7) | 3.0 (0.6) |
| Day 1 | 3.1 (0.6) | 2.8 (0.6) |
| Day 14 | 3.3 (0.7) | 3.0 (0.6) |
| Day 15 | 3.3 (0.7) | 2.8 (0.7) |
| Day 22 | 3.3 (0.7) | 3.0 (0.6) |
| PEF (AM, L/min) | | |
| Baseline | 405.5 (91) | 365.1 (93) |
| Day 22 | 423.4 (89.5) | 375.3 (97) |
| Rescue free days (%) | | |
| Baseline | 40.9 (41) | 39 (39) |
| Day 22 | 68.4 (32) | 63.5 (33) |
| Doses of Rescue Rx | | |
| Baseline | 17 (15) | 14.7 (11) |
| Day 22 | 3.5 (4) | 3.9 (4.5) |
| PC20 (mg/ml) | | |
| Baseline | 1.4 (1.3) | 3.5 (5.7) |
| Day 22 | 2.0 (1.6) | 4.0 (8.9) |
| ACSS | | |
| Baseline | 73.1 (14) | 65 (11.1) |
| Day 22 | 82.4 (8.4) | 71.5 (16.3) |

TABLE 2b
Efficacy Inflammatory parameters

| | Budesonide (SD) | TPI 1020 (SD) |
|---|---|---|
| eNO (ppb) | | |
| Baseline | 19 (17.6) | 29 (27) |
| Day 22 | 9.8 (7.6) | 16.9 (14) |
| Sputum eos (%) | | |
| Baseline | 2.4 (3.3) | 8.7 (10.8) |
| Day 22 | 1.0 (1.0) | 3.3 (4.0) |
| Blood eos (%) | | |
| Baseline | 2.3 (2.3) | 2.8 (2.7) |
| Day 22 | 2.1 (1.6) | 1.8 (1.7) |
| Blood lymphocytes | | |
| (%) | | |
| Baseline | 14.9 (13) | 20.3 (16.6) |
| Day 22 | 22.5 (15.8) | 18.4 (15.4)* |
| CRP (mg/L) | | |
| Baseline | 2.7 (4.3) | 3.6 (3.9) |
| Day 22 | 2.5 (1.4) | 3.0 (4.5) |

There was no improvement in the FEV1 in both groups over the 3 week period, doses of rescue medication, PC₂₀, and the ACSS score improved to a similar extent in the budesonide and the TPI-1020 groups. Sputum eosinophils, blood eosinophils and eNO tended to be higher at baseline in the TPI-1020 group but improved to the same extent as in the budesonide group. Blood lymphocytes increased in budesonide treated patients, a difference that was significantly different when compared to TPI-1020 treated patients (p<0.05).

Plasma CRP decreased more in TPI-1020 treated patients on day 15 versus baseline (increase by 0.7 in budesonide group versus decrease by 1.4 mg/L in TPI-1020 group, p=0.03). On day 22, CRP levels were not significantly different between groups (p=0.09), one patient in the TPI-1020 group having an 8.5 fold increase in the CRP level attributed to a viral infection without an exacerbation of asthma. When compared to baseline, 22 days of therapy decreased CRP in 33.2% of patients in the budesonide group and 69.2% of patients in the TPI-1020 group.

### Discussion:

In this study we have found that NO-donating budesonide derivatives of the present invention, such as TPI-1020, appear to be safe in patients with asthma and has similar effects as equimolar doses of budesonide on FEV₁, PEF, PC₂₀, use of rescue medication, eNO and eosinophilic inflammation. These compounds have additional beneficial effects that seem to be NO-related on sputum neutrophils, blood pressure and serum CRP. Taken together, in association with a lower systemic budesonide exposure and lack of effect on 24 hour urinary free cortisol, these compounds are potential drug candidates not only for allergic airway inflammation but also for diseases where neutrophils and systemic inflammation seem to play a role.

Asthma is characterized by increased airway inflammation and elevated levels of exhaled NO (eNO). Allergen challenge aggravates asthma and leads to an increase in eNO. When asthma becomes controlled with corticosteroid therapy, eNO decreases, often to a level that is found in normal individuals. These results have lead to speculation that NO is negatively influencing the ongoing inflammation within the airways of patients with asthma. However, NO has been shown to have anti-viral and anti-bacterial effects, cause bronchodilation in patients with asthma and over-expression of eNO is beneficial in a mouse model of allergic airway inflammation. We assessed whether NO-donating budesonide derivatives such as TPI-1020 would be safe in asthma. We chose smokers with asthma since this would also permit us to assess whether such compounds would affect sputum neutrophils in these patients.

We noted TPI-1020, being exemplary of the compounds of the present invention, was safe in patients with asthma over the three weeks of administration, even at the highest dose that would be clinically relevant (equivalent to 1600 mcg of budesonide per day). With TPI-1020, we noted three fold less treatment emergent adverse events and less adverse events that were considered by the investigator to be drug related. Inhaled corticosteroids are considered safe at low doses in patients with asthma, but medium (800 mcg equivalent of budesonide per day) and high doses (1600 mcg equivalent) have been associated with long term toxicity. These findings have led to the recommendation that add on therapy be considered when a patient with asthma is not controlled on low dose inhaled corticosteroids.

One of the measures that has been shown to help predict the potential for long term toxicity of corticosteroids is the 24 hour urinary free cortisol measurement (ref UFC). A dose response relationship between the dose of budesonide administered and UFC has been reported. In this study we have confirmed that 1600 mcg of budesonide per day decreases significantly UFC. TPI-1020 had no significant effect on UFC at a dose that was equimolar to 1600 mcg of budesonide per day. More studies are needed to understand the absorption, metabolism and distribution of these compounds in humans but the absence of effect of TPI-1020 on UFC may be related to the different plasma pharmacokinetic profile of budesonide in both treatment groups. Indeed, the Cmax and AUC of plasma budesonide was more than five fold lower in the NO-donating budesonide derivative group than in the equimolar budesonide group. It could thus be expected that less systemic exposure would translate to less effects on the adrenal-pituitary axis.

Although this study was designed to assess safety, we did perform common measures associated with efficacy. The length of the study was not long enough to see the maximal corticosteroid effects and the power of the study was not sufficient to draw any significant conclusions on these standard measures. However, the compounds of the present invention seemed to have the same trends in effects on FEV₁, PEF, days with rescue medication usage, the amount of rescue medication employed, asthma control scoring system, methacholine response, sputum eosinophils and exhaled NO. It is interesting that TPI-1020, for example, decreased to a similar extent eNO as the equimolar amount of budesonide (12.1 versus 9.2 ppb decrease with TPI-1020 versus. budesonide, respectively). These results would suggest that the amount of NO released by TPI-1020 within the lungs is not sufficient to affect eNO or that the NO released is metabolised prior to making it into the air that is present within the airways.

We assessed whether the NO released by the compounds of the present invention would have pharmacological effects within the lungs. NO has been shown to inhibit neutrophil recruitment and compounds like TPI-1020 inhibited the recruitment of neutrophils into the lungs of a guinea pig model of LPS induced lung inflammation, whilst budesonide was without effect in this model. In our study, TPI-1020 decreased neutrophils in the sputum of patients with asthma after 22 days by 32.6%, whereas budesonide had no effect. Although we had powered the study for a 20% difference in sputum neutrophils between groups, the difference reported was not significantly different. We believe that this could be explained by the unexpectedly large number of patients with normal neutrophils at baseline. Indeed 6 of 13 TPI-1020-treated patients had sputum neutrophils at baseline below 20%. Interestingly, the decrease in sputum neutrophils caused by TPI-1020 occurred only in the patients with high neutrophils at baseline suggesting that NO-donating budesonide derivatives such as TPI-1020 are acting on the mechanism by which cigarette smoke recruits neutrophils into the airways and not on the normal homeostatic mechanism of neutrophil recruitment.

Two additional pharmacological effects that may be NO-related were found in this study. We noted that compounds such as TPI-1020 affected measures of blood pressure at 15 and 22 days. The effects were mostly on systolic blood pressure and on the pulse pressure. Although nitrates are known to decrease blood pressure, a decrease in blood pressure has not been previously reported with inhaled NO. Inhaled NO has been shown to improve pulmonary hypertension in patients with COPD over a 3-month period without any effects on systemic blood pressure. Because of the effects on systemic blood pressure, the compounds of the present invention may have additional long term benefits in patients with hypertension or with COPD where systolic blood pressure is increased and pulse pressure is increased. Increased serum CRP has been associated with increased mortality in patients with cardiovascular diseases and with COPD. Inhaled corticosteroids have been shown to decrease CRP but TPI-1020 decreased CRP more than equimolar doses of budesonide. These effects may be caused by the additional anti-inflammatory effects of NO on chemokines and cytokines.

In conclusion, we have found that NO-donating budesonide derivatives of the present invention, such as TPI-1020, appear to be safe in patients with asthma. In addition to having similar effects in asthma as equimolar doses of budesonide, these compounds have an improved pharmacokinetic profile with no effects on UFC and additional effects on sputum neutrophils, blood pressure and CRP that would make a novel therapeutic option for diseases of the lungs where increased neutrophils and systemic inflammation play a role. Importantly, the data presented herein demonstrated that when patients inhale budesonide there was a significant amount of drug in the blood as reflected in relatively high AUC. In contrast, when patients inhaled TPI-1020 there was much less steroid in the blood as indicated by the significantly lower AUC values. This lower AUC is surprising since the steroid load to the patients in the two groups was comparable. Accordingly, it would appear that the compounds as contemplated for use in the present invention provide for an improved pulmonary retention with less systemic effects thereby rendering a considerably safer respiratory steroid therapy than the prior art.

These results suggest that nitric oxide donating compounds, and more specifically nitric oxide donating corticosteroids of the present invention represent a new promising therapy to treat respiratory diseases.

The foregoing is provided for purposes of illustrating, explaining, and describing embodiments of this invention. Modifications and adaptations to these embodiments will be apparent to those skilled in the art and may be made without departing from the scope or spirit of this invention.

1. A method for treating a respiratory disease in patients comprising administering a therapeutically effective amount of a compound having Formula 3:
A-W 3
wherein A is a steroid residue, and
W is any nitric oxide (“NO”) donating moiety linked to the steroid backbone and is capable of donating, releasing and/or directly or indirectly transferring any of the three redox forms of nitrogen monoxide (NO⁺, NO⁻, NO.) such that there is a statistically significant decrease in blood pressure of the patients, said compound.

2. The method of claim 1 wherein steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

3. The method of claim 1, wherein the compound has Formula (4): 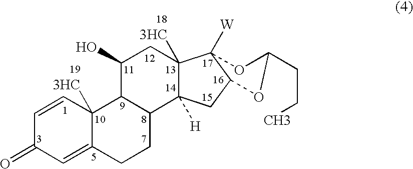
wherein W is any nitric oxide (“NO”) donating moiety attached thereto capable of donating, releasing and/or directly or indirectly transferring any of the three redox forms of nitrogen monoxide (NO⁺, NO⁻, NO.).

4. The method of claim 1 wherein W is selected from Formulae A-G, wherein
Formula A compounds being represented by: —C(O)-L-(X_{O})—(X₁)—HNO₂ (as described in published U.S. Patent Application Publication No. 2006/0052594 which is hereby incorporated herein by reference thereto) where L is defined as:
(CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;
X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and
X₁ is a bivalent-linking group selected from the following: 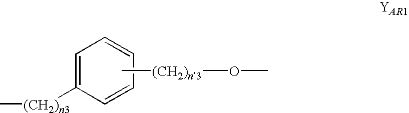
wherein n3 is an integer from 0 to 5 and n3′ is an integer from 1 to 3; 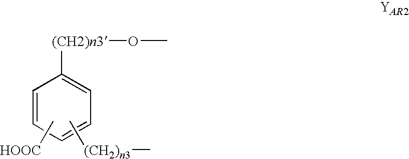
wherein n3 and n3′ have the above meaning or 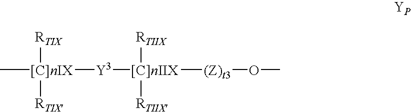
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning: 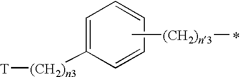
wherein:
* shows the position of the ONO₂ group;
T has the following meanings:
—COX₃—, —X₃CO—, wherein X₃═S or X_{O} as above defined;
—X₃— as above defined;
n3 and n′3 are as above defined; or
Y³ is selected from the following bivalent radicals: 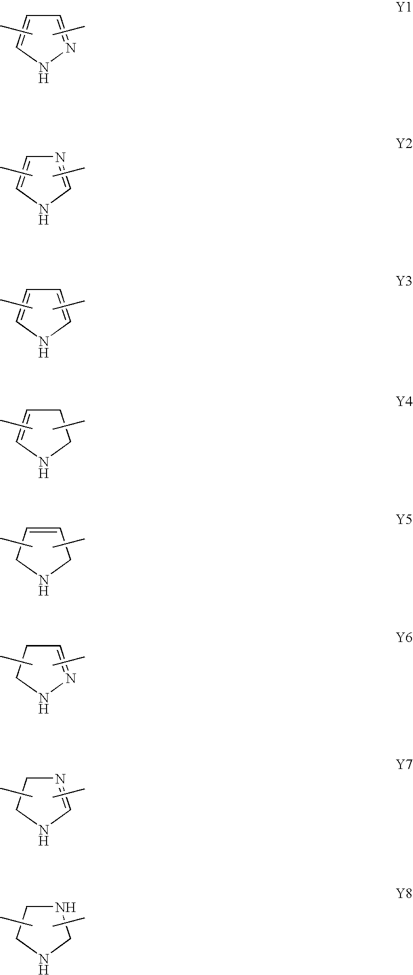 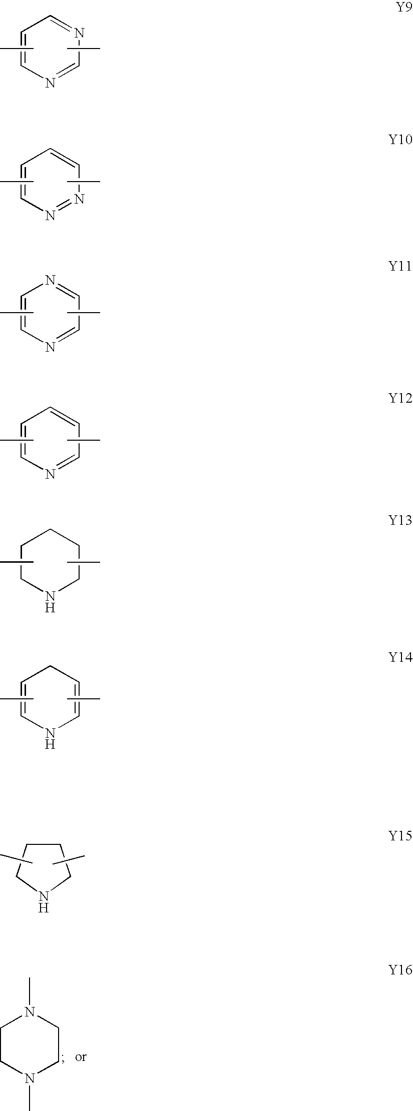
(Y12), having the two free valences in the ortho positions with respect to the nitrogen atom; (Y16) with the two valences linked to the two heteroatoms, (Y1) (pyrazol) 3,5-disubstituted; or (Y16); or
L is na=n′b=1, n′a=2, n″b=n′″b=n″a=nb=0, R₄═CH₃, R₅═R_{4″}═R_{5″}═H, where the precursors of the bivalent radicals X₁, as defined, or A is:
—(CO-L)ₜ-(X)ₜ₁—X₁—NO₂
where t and t1 are integers=1 and L and X are defined as above as L and X₀, but where
X₁ is a bivalent-connecting bridge is selected from the group consisting of Y—O and Y_{1,}, wherein:
for Y—O, Y is a linear or whenever possible branched C₁-C₂₀ alkylene, preferably having from 2 to 5 carbon atoms, or an optionally substituted cycloalkylene having from 5 to 7 carbon atoms; and
for Y_{1,} Y is selected from Y_{AR1}, Y_{AR2}, and Y_{P}, as defined above, and is more particularly: 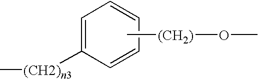
where n3 is an integer from 0 to 3; 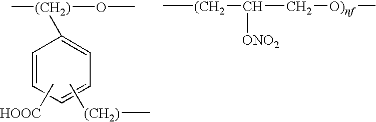
where nf′ is an integer from 1 to 6, preferably from 2 to 4; or 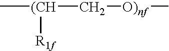
where R_{1f}═H, CH₃ and nf is an integer from 1 to 6, preferably from 2 to 4; or
A is —(CO-L)ₜ-(X)ₜ₁—X₁—NO₂
where t and t1 are integers=1 and L is defined as above;
wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;
X is equal to O, C═O, NH, NR_{1c} where R_{1c} is a C₁-C₁₀, and preferably a C₁-C₄ linear or branched alkyl; OH, CH₃, Cl, N(—CH₂—CH₃)₂, SCH₂F, SH, 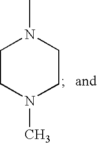
X₁ is a bivalent-connecting bridge is selected from the group consisting of Y—O and Y₁ as defined above; or
A is represented as
C(O)-L-(X₀)—(X₁)—NO₂
wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a} wherein na and nb=1; R₄ and R₅═H; and wherein n′a, n″a, n′b, n″b and n′″b are 0 (thus L is CH₂O.)
X₀ is C═O, and
X₁ is the bivalent-linking group Y_{AR1}, preferably 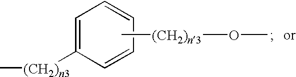
Formula B compounds being represented by: —C(O)CH₂O—X_{Z} or alternatively by —C(O)CH₂OC(O)—X_{Z} wherein X_{Z} is defined as follows: 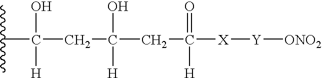
and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃; 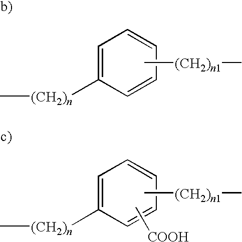
wherein for b) and c) above n is an integer from 0 to 20, and n¹ is an integer from 0 to 20; 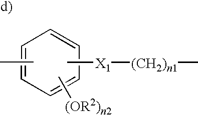
wherein, n¹ is as defined above and n² is an integer from 0 to 2; X₁ is —OCO— or —OCO— and R₂ is H or CH₃; 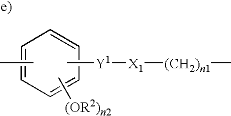
wherein n¹, n², R₂ and X₁ are as defined above; and Y¹ is either —CH₂—CH₂— or —CH₂═CH₂—(CH₂)ₙ₂—; 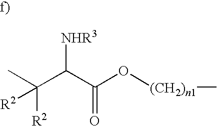
wherein n¹ and R² are as defined above; R³ is H or COCH₃; with the proviso that when Y is selected from the bivalent radicals mentioned under b) through f), the, —ONO₂ group is bound to —(CH₂)ₙ₁; 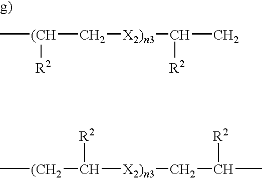
wherein X₂ is O or S, n³ is an integer from 1 to 6, preferably from 1 to 4, and R² is defined above; 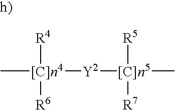
wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵, R⁶, and R⁷ are H; wherein the —ONO₂ group is bound to the following structure: 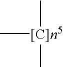
wherein, n⁵ is defined above;
Y² is a heterocyclic saturated, unsaturated or aromatic 5 or 6 members ring, containing one or more heteroatoms selected from nitrogen, oxygen, sulfur, and is selected from the following structures H1 through H13: 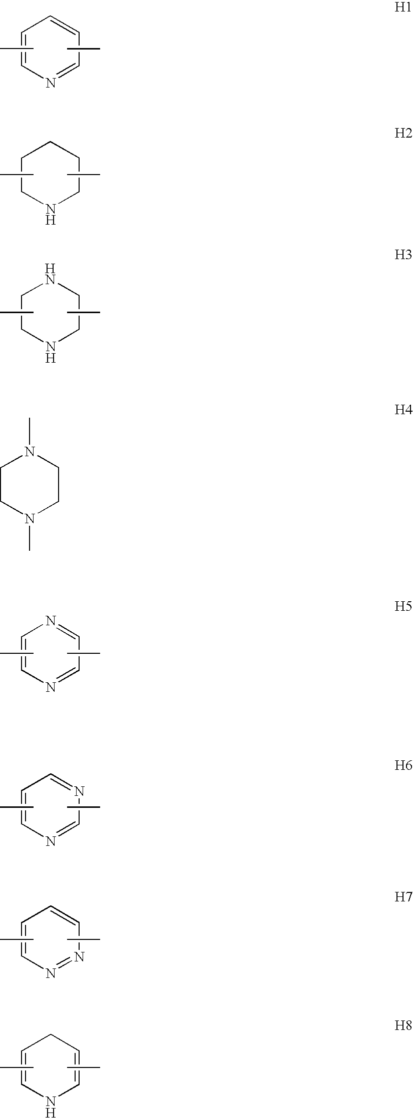 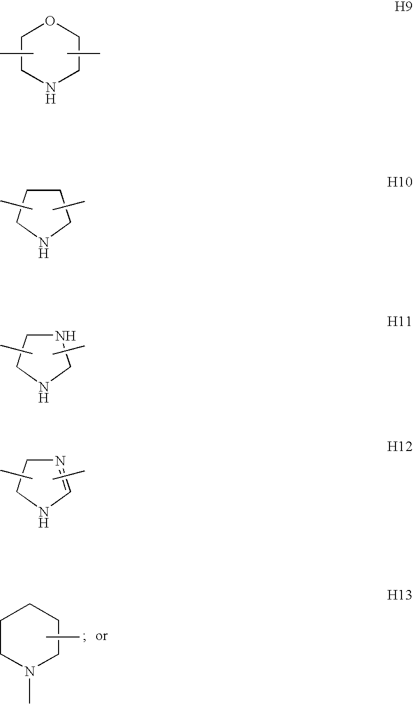
Formula C compounds being represented by: —C(O)CH₂O—(X₁—ONO₂)ₛ or alternatively by —C(O)CH₂OC(O)—(X₁—ONO₂)ₛ wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—;
Formula D compounds being represented by: —C(O)CH₂O—B—C) wherein X₁ is defined as follows: 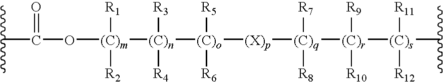
where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above;
Formula E compounds being represented by: —C(O)CH₂O—B—C^{Z}, where C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position;
wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
B—C^{Z} is equal to the following structure: 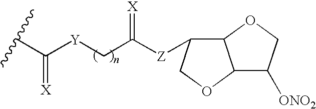
wherein n is an integer from 1 to 4; X═O or S; Y=methylene, O, or NH₂; and Z=O or NH₂; or where
W=Formula E′ where E′ is —C(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position; or
Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
R₁ can be selected from any of the following chemical moieties that have been appropriately substituted with a NO donating group: lower alkyls/alkenyls/alkynyls; that are substituted or unsubstituted; substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.
Formula F compounds being represented by: —C(O)CH₂O—K or alternatively by —C(O)CH₂OC(O)—K wherein K is defined as follows:
—Y—(CR₄R_{4′})ₚ-T-(CR₄R_{4′})ₚ—ONO₂;
—Y—(CR₄R_{4′})ₒ-[phenyl]-T-(CR₄R_{4′})ₚ—ONO₂;
wherein T is ortho, meta or para;
—Y—B-[piperazinyl]-W—(CR₄R_{4′})ₚ—ONO₂;
—Y—(CR₄R_{4′})ₚ—V—B-T-(CR₄R_{4′})ₚ—ONO₂;
—Y—(CR₄R_{4′})ₚ-T-C(O)—(CR₄R_{4′})ₚ—(CH₂)—ONO₂;
—Y—(CR₄R_{4′})ₚ—C(Z)-(CH₂)_{q}-T-(CR₄R_{4′})_{q}—(CH₂)—ONO₂;
—Y—(CR₄R_{4′})-T-(CH₂)_{q}—V—(CR₄R_{4′})_{q}—(CH₂)—ONO₂;
—Y—(CR₄R_{4′})ₚ—V—(CH₂)_{q}—V—(CR₄R_{4′})_{q}—(CH₂)—ONO₂;
—Y—(CR₄R_{4′})ₒ—(W)_{q}—(CH₂)_{q}—V—(CR₄R_{4′})ₒ—(CH₂)—ONO₂;
—NRⱼ—O—(CH₂)ₒ—V—(CR₄R_{4′})_{q}—(CH₂)—ONO₂;
—NRⱼ—O—(CH₂)ₒ—(W)_{q}—(CR₄R_{4′})_{q}—(CH₂)—ONO₂;
—O—NRⱼ—(CH₂)ₒ—(W)_{q}—(CR₄R_{4′})_{q}—(CH₂)—ONO₂;
Formula G compounds being represented by: —C(O)CH₂O—X1 or alternatively by —C(O)CH₂OC(O)—X1 wherein X1 is defined as follows:
—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;
or alternatively, X1 is equal to K which is further defined as:
—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);
where, —(U)—(V) is equal to —Wᵢ-Eⱼ-W_{g}-[C—(Rₑ)(R_{f})]_{z}-T-Q; or alternatively
U and V are taken independently wherein U is O, S, or —N(Rₐ)(Rᵢ) and V is nitro, nitroso, or hydrogen;
wherein, a, b, c, d, g, i and j are each independently an integer from 0 to 3;
p, x, y and z are each independently an integer from 0 to 10;
Wₐ, W_{d}, Wᵢ, and W_{g} are independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ)—;
o is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group, wherein, in cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

5. The method of claims 1, wherein the compound has the structure of Formula 1: 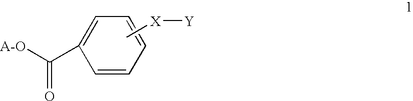
wherein A is a steroid residue;
X is a C₁-C₅ branched or linear chain alkyl; and
Y is either (ONO₂) or (ONO); with the proviso that A is linked to the remainder of Formula 1 by way of the C-11 or the C-17 position.

6. The method of claim 5, wherein the steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

7. The method of claim 5, wherein A is budesonide, X is CH₂ and Y is (ONO₂) and A is linked to the remainder of Formula 1 through the C-17 position.

8. The method of claim 5, wherein the steroid residue is a corticosteroid budesonide and the NO-donor is linked to budesonide to provide a compound of Formula 2:

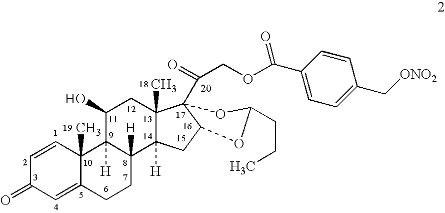

9. The method of claim 1, wherein the respiratory disease is selected from the group consisting of asthma, chronic obstructive pulmonary disorder, eosinophilic cough, bronchitis, acute and chronic rejection of lung allograft, sarcoidosis, pulmonary fibrosis, rhinitis, pulmonary hypertension, cystic fibrosis, bronchiectasis and sinusitis.

10. The method of claim 9, wherein the respiratory disease is asthma.

11. The method of claim 9, wherein the respiratory disease is chronic obstructive pulmonary disorder.

12. The method of claim 1, wherein the compound is administered via inhalation at a dose from about 100 μg to about 3000 μg.

13. The method of claim 12, wherein the compound is administered via inhalation at a dose from about 200 μg to about 2400 μg.

14. The use according to claim 12, wherein the compound is administered via inhalation at a dose from about 400 μg to about 1500 μg.

15. The method of claim 12, wherein the compound is administered via inhalation at a dose from about 600 μg to about 1200 μg.

16. The method of claim 12, wherein the compound is administered via inhalation at a dose from about 800 μg to about 1200 μg.

17. The method of claim 1, wherein the compound is administered twice daily.

18. The method of claim 1, wherein the compound is administered as needed.

19. The method of claim 1 wherein the compound is administered orally at a dose from about 100 μg to about 250 mg.

20. The method of claim 1 wherein the compound is administered intravenously at a dose from about 100 μg to about 1 g.

21. A method for providing a statistically significant decrease in sputum neutrophil level in patients having respiratory disease treated with a steroidal compound, said compound having Formula 3:
A-W 3
wherein A is a steroid residue, and
W is any nitric oxide (“NO”) donating moiety linked to the steroid backbone and is capable of donating, releasing and/or directly or indirectly transferring any of the three redox forms of nitrogen monoxide (NO⁺, NO⁻, NO.).

22. The method of claim 21 wherein steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

23. The method of claim 21, wherein the compound has Formula (4): 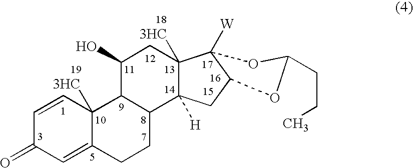
wherein W is any nitric oxide (“NO”) donating moiety attached thereto capable of donating, releasing and/or directly or indirectly transferring any of the three redox forms of nitrogen monoxide (NO⁺, NO⁻, NO.).

24. The method of claim 21 wherein W is selected from Formulae A-G, wherein
Formula A compounds being represented by: —C(O)-L-(X_{O})—(X₁)—NO₂ (as described in published U.S. Patent Application Publication No. 2006/0052594 which is hereby incorporated herein by reference thereto) where L is defined as:
(CR₄R₅)ₙₐ(O)_{nb}(CR_{4′}R_{5′})ₙₐ(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;
X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and
X₁ is a bivalent-linking group selected from the following: 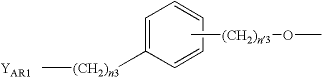
wherein n3 is an integer from 0 to 5 and n3′ is an integer from 1 to 3; 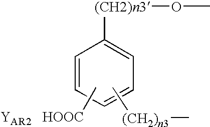
wherein n3 and n3′ have the above meaning or 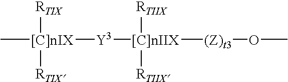
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning: 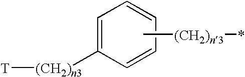
wherein:
* shows the position of the ONO₂ group;
T has the following meanings:
—COX₃—, —X₃CO—, wherein X₃═S or X_{O} as above defined;
—X₃— as above defined;
n3 and n′3 are as above defined; or
Y³ is selected from the following bivalent radicals: 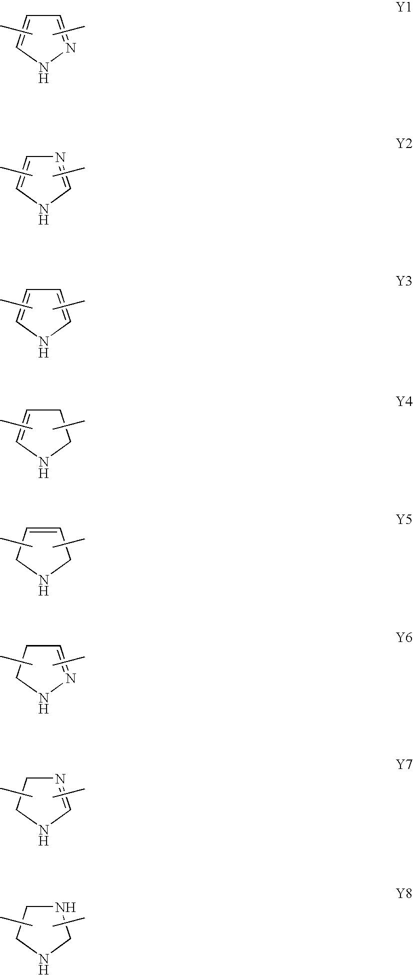 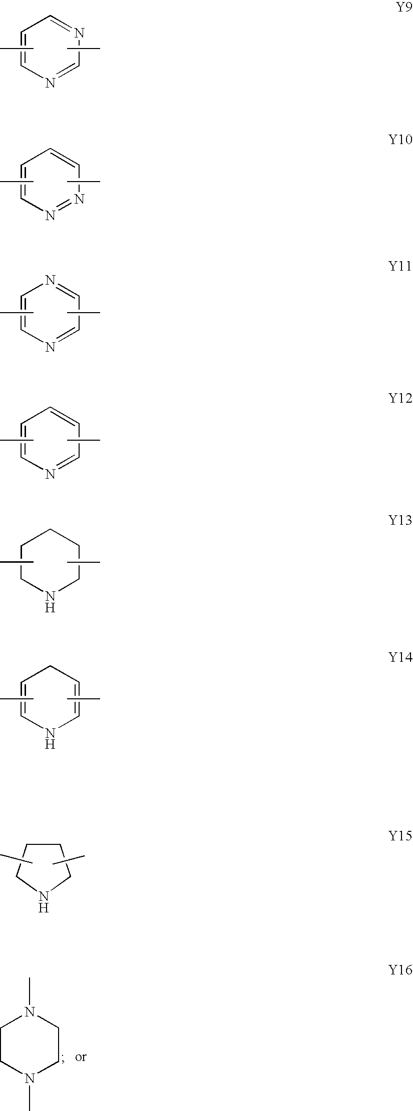
(Y12), having the two free valences in the ortho positions with respect to the nitrogen atom; (Y16) with the two valences linked to the two heteroatoms, (Y1) (pyrazol) 3,5-disubstituted; or (Y16); or
L is na=n′b=1, n′a=2, n″b=n′″b=n″a=nb=0, R₄═CH₃, R₅═R₄═R₅═H, where the precursors of the bivalent radicals X₁, as defined, or A is:
—(CO-L)ₜ-(X)ₜ₁—X₁—NO₂
where t and t1 are integers=1 and L and X are defined as above as L and X₀, but where
X₁ is a bivalent-connecting bridge is selected from the group consisting of Y—O and Y_{1,}, wherein:
for Y—O, Y is a linear or whenever possible branched C₁-C₂₀ alkylene, preferably having from 2 to 5 carbon atoms, or an optionally substituted cycloalkylene having from 5 to 7 carbon atoms; and
for Y₁, Y is selected from Y_{AR1}, Y_{AR2}, and Y_{P}, as defined above, and is more particularly: 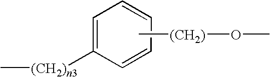
where n3 is an integer from 0 to 3; 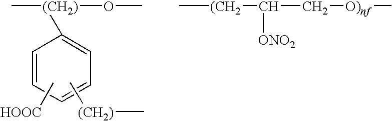
where nf′ is an integer from 1 to 6, preferably from 2 to 4; or 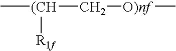
where R_{1f}═H, CH₃ and nf is an integer from 1 to 6, preferably from 2 to 4; or
A is —(CO-L)ₜ-(X)ₜ₁—X₁—NO₂
where t and t1 are integers=1 and L is defined as above;
wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;
X is equal to O, C═O, NH, NR_{1c} where R₁ is a C₁-C₁₀, and preferably a C₁-C₄ linear or branched alkyl; OH, CH₃, Cl, N(—CH₂—CH₃)₂, SCH₂F, SH, 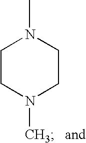
X₁ is a bivalent-connecting bridge is selected from the group consisting of Y—O and Y₁ as defined above; or
A is represented as
C(O)-L-(X₀)—(X₁)—NO₂
wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)
X₀ is C═O, and
X₁ is the bivalent-linking group Y_{AR1}, preferably 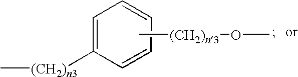
Formula B compounds being represented by: —C(O)CH₂O—X_{Z} or alternatively by —C(O)CH₂OC(O)—X_{Z} wherein X_{Z} is defined as follows: 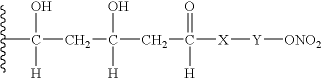
and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃; 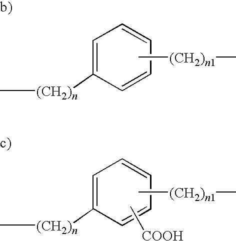
wherein for b) and c) above n is an integer from 0 to 20, and n¹ is an integer from 0 to 20; 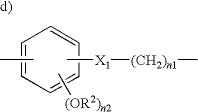
wherein, n¹ is as defined above and n² is an integer from 0 to 2; X₁ is —OCO— or —OCO— and R₂ is H or CH₃; 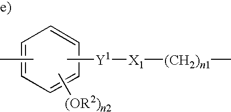
wherein n¹, n², R₂ and X₁ are as defined above; and Y¹ is either —CH₂—CH₂— or —CH₂═CH₂—(CH₂)ₙ₂—; 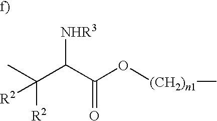
wherein n¹ and R² are as defined above; R³ is H or COCH₃; with the proviso that when Y is selected from the bivalent radicals mentioned under b) through f), the, —ONO₂ group is bound to —(CH₂)ₙ₁; 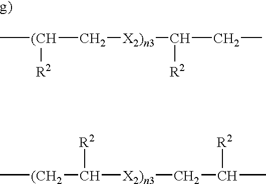
wherein X₂ is O or S, n³ is an integer from 1 to 6, preferably from 1 to 4, and R² is defined above; 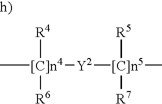
wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵, R⁶, and R⁷ are H; wherein the —ONO₂ group is bound to the following structure: 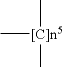
wherein, n⁵ is defined above;
Y² is a heterocyclic saturated, unsaturated or aromatic 5 or 6 members ring, containing one or more heteroatoms selected from nitrogen, oxygen, sulfur, and is selected from the following structures H1 through H13: 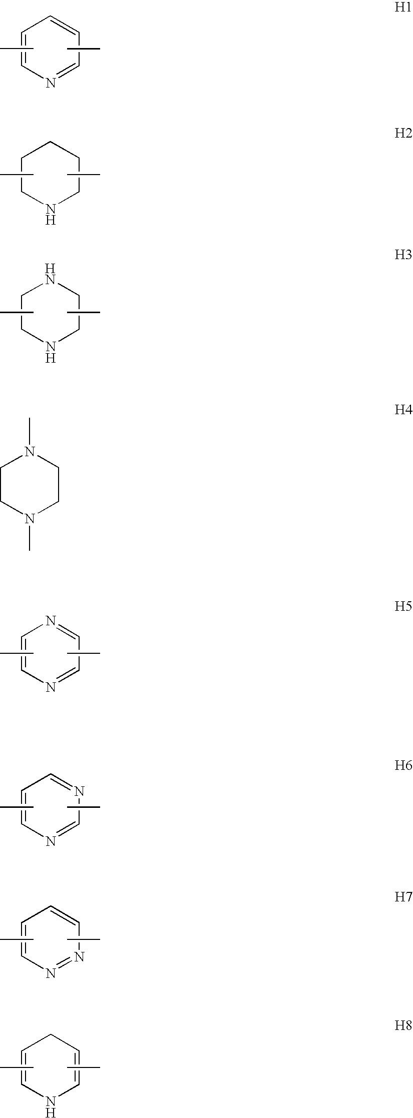 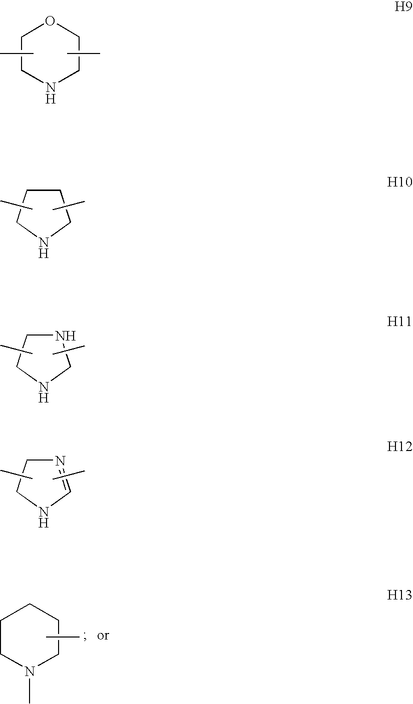
Formula C compounds being represented by: —C(O)CH₂O—(X₁—ONO₂)ₛ or alternatively by —C(O)CH₂OC(O)—(X₁—ONO₂)ₛ wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—;
Formula D compounds being represented by: —C(O)CH₂O—B—C) wherein X₁ is defined as follows: 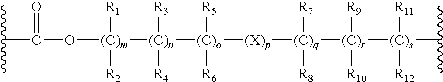
where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above;
Formula E compounds being represented by: —C(O)CH₂O—B—C^{Z}, where C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position;
wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
B—C^{Z} is equal to the following structure: 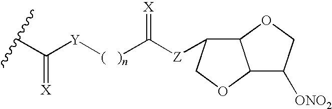
wherein n is an integer from 1 to 4; X═O or S; Y=methylene, O, or NH₂; and Z=O or NH₂; or where
W=Formula E′ where E′ is —C(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position; or
Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
R₁ can be selected from any of the following chemical moieties that have been appropriately substituted with a NO donating group: lower alkyls/alkenyls/alkynyls; that are substituted or unsubstituted; substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.
Formula F compounds being represented by: —C(O)CH₂O—K or alternatively by —C(O)CH₂OC(O)—K wherein K is defined as follows:
—Y—(CR₄R_{4′})ₚ-T-(CR₄R_{4′})ₚ—ONO₂;
—Y—(CR₄R_{4′})ₒ-[phenyl]-T-(CR₄R_{4′})ₚ—ONO₂;
wherein T is ortho, meta or para;
—Y—B-[piperazinyl]-W—(CR₄R_{4′})ₚ—ONO₂;
—Y—(CR₄R_{4′})ₚ—V—B-T-(CR₄R_{4′})ₚ—ONO₂;
—Y—(CR₄R_{4′})ₚ-T-C(O)—(CR₄R_{4′})ₚ—(CH₂)—ONO₂;
—Y—(CR₄R_{4′})ₚ—C(Z)-(CH₂)_{q}-T-(CR₄R_{4′})_{q}—(CH₂)—ONO₂;
—Y—(CR₄R_{4′})-T-(CH₂)_{q}—V—(CR₄R_{4′})_{q}—(CH₂)—ONO₂;
—Y—(CR₄R_{4′})ₚ—V—(CH₂)_{q}—V—(CR₄R_{4′})_{q}—(CH₂)—ONO₂;
—Y—(CR₄R_{4′})ₒ—(W)_{q}—(CH₂)_{q}—V—(CR₄R_{4′})ₒ—(CH₂)—ONO₂;
—NRⱼ—O—(CH₂)ₒ—V—(CR₄R_{4′})_{q}—(CH₂)—ONO₂;
—NRⱼ—O—(CH₂)ₒ—(W)_{q}—(CR₄R_{4′})_{q}—(CH₂)—ONO₂;
—O—NRⱼ—(CH₂)ₒ—(W)_{q}—(CR₄R_{4′})_{q}—(CH₂)—ONO₂;
Formula G compounds being represented by: —C(O)CH₂O—X1 or alternatively by —C(O)CH₂OC(O)—X1 wherein X1 is defined as follows:
—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;
or alternatively, X1 is equal to K which is further defined as:
—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);
where, —(U)—(V) is equal to —Wᵢ-Eⱼ-W_{g}-[C—(Rₑ)(R_{f})]_{z}-T-Q; or alternatively
U and V are taken independently wherein U is O, S, or —N(Rₐ)(Rᵢ) and V is nitro, nitroso, or hydrogen;
wherein, a, b, c, d, g, i and j are each independently an integer from 0 to 3;
p, x, y and z are each independently an integer from 0 to 10;
Wₐ, W_{d}, Wᵢ, and W_{g} are independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ)—;
o is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group, wherein, in cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

25. The method of claim 21, wherein the compound has the structure of Formula 1: 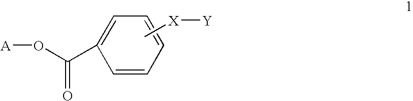
wherein A is a steroid residue;
X is a C₁-C₅ branched or linear chain alkyl; and
Y is either (ONO₂) or (ONO); with the proviso that A is linked to the remainder of Formula 1 by way of the C-11 or the C-17 position.

26. The method of claim 25, wherein the steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

27. The method of claim 25, wherein A is budesonide, X is CH₂ and Y is (ONO₂) and A is linked to the remainder of Formula 1 through the C-17 position.

28. The method of claim 25, wherein the steroid residue is a corticosteroid budesonide and the NO-donor is linked to budesonide to provide a compound of Formula 2:

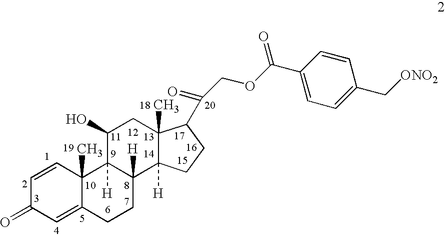

29. The method of claim 21, wherein the respiratory disease is selected from the group consisting of asthma, chronic obstructive pulmonary disorder, eosinophilic cough, bronchitis, acute and chronic rejection of lung allograft, sarcoidosis, pulmonary fibrosis, rhinitis, pulmonary hypertension, cystic fibrosis, bronchiectasis and sinusitis.

30. The method of claim 29, wherein the respiratory disease is asthma.

31. The method of claim 29, wherein the respiratory disease is chronic obstructive pulmonary disorder.

32. The method of claim 21, wherein the compound is administered via inhalation at a dose from about 100 μg to about 3000 μg.

33. The method of claim 32, wherein the compound is administered via inhalation at a dose from about 200 μg to about 2400 μg.

34. The method of claim 32, wherein the compound is administered via inhalation at a dose from about 400 μg to about 1500 μg.

35. The method of claim 32, wherein the compound is administered via inhalation at a dose from about 600 μg to about 1200 μg.

36. The method of claim 32, wherein the compound is administered via inhalation at a dose from about 800 μg to about 1200 μg.

37. The method of claim 21, wherein the compound is administered twice daily.

38. The method of claim 21, wherein the compound is administered as needed.

39. The method of claim 21 wherein the compound is administered orally at a dose from about 100 μg to about 250 mg.

40. The method of claim 21 wherein the compound is administered intravenously at a dose from about 100 μg to about 1 g.

41. A method for providing a statistically significant decrease in plasma CRP level in patients having a respiratory disease said compound having Formula 3:
A-W 3
wherein A is a steroid residue, and
W is any nitric oxide (“NO”) donating moiety linked to the steroid backbone and is capable of donating, releasing and/or directly or indirectly transferring any of the three redox forms of nitrogen monoxide (NO⁺, NO⁻, NO.).

42. The method of claim 41 wherein steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

43. The method of claim 41, wherein the compound has Formula (4): 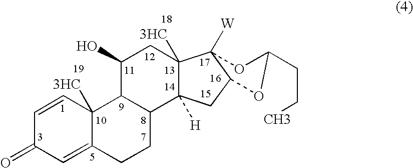
wherein W is any nitric oxide (“NO”) donating moiety attached thereto capable of donating, releasing and/or directly or indirectly transferring any of the three redox forms of nitrogen monoxide (NO⁺, NO⁻, NO.).

44. The method of claim 41 wherein W is selected from Formulae A-G, wherein
Formula A compounds being represented by: —C(O)-L-(X_{O})—(X₁)—NO₂ (as described in published U.S. Patent Application Publication No. 2006/0052594 which is hereby incorporated herein by reference thereto) where L is defined as:
(CR₄R₅)ₙₐ(O)_{nb}(CR_{4′}R_{5′})ₙₐ(CO)_{n′b}(O)_{n″b}(CO)_{n′″b}(CR_{4″}R_{5″})_{n″a}, wherein na and nb=1; R₄ and R₅═H; and wherein n′a, and n″a, equal to or different from each other, are integers from 0 to 6, preferably 1-3; n′b, n″b and n′″b, equal to or different from each other, are integers equal to 0 or 1, R_{4′}, R_{5′}, R_{4″}, R_{5″}, equal to or different from each other, are selected from H, C₁-C₅, preferably C₁-C₃ linear or branched alkyl;
X_{O}═O, C═O, NH, NR_{1c} wherein R_{1c} is a C₁-C₁₀, and preferably a C₁-C₄ linear, branched, or cyclic alkyl; the bond between the steroid backbone and the linking group X₁ is ester or amidic type, and
X₁ is a bivalent-linking group selected from the following: 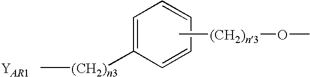
wherein n3 is an integer from 0 to 5 and n3′ is an integer from 1 to 3; 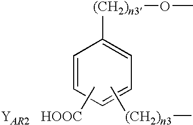
wherein n3 and n3′ have the above meaning or 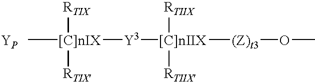
wherein:
nIX is an integer from 0 to 10, preferably 1-3;
nIIX is an integer from 1 to 10, preferably 1-5;
R_{TIX}, R_{TIX′}, R_{TIIX}, R_{TIIX′}; equal to or different from each other are H or C₁-C₄ linear or branched alkyl; preferably R_{TIX}, R_{TIX′}, R_{TIIX}, R_{TIIX′} are H;
Y³ is a saturated, unsaturated or aromatic heterocyclic ring, having 5 or 6 atoms, containing from one to three heteroatoms, preferably from one to two, said heteroatoms being equal or different and selected from nitrogen, oxygen, sulphur; preferably nitrogen;
t3 is zero or 1;
Z has the following meaning: 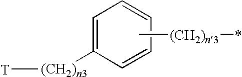
wherein:
* shows the position of the ONO₂ group;
T has the following meanings:
—COX₃—, —X₃CO—, wherein X₃═S or X_{O} as above defined;
—X₃— as above defined;
n3 and n′3 are as above defined; or
Y³ is selected from the following bivalent radicals: 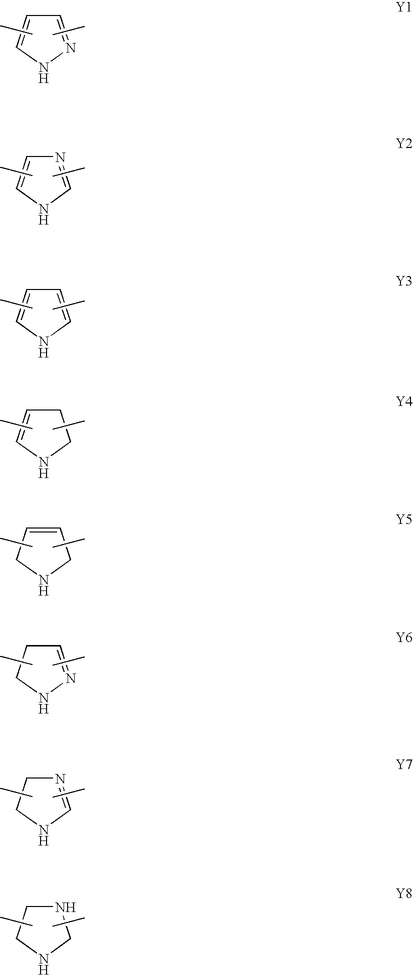 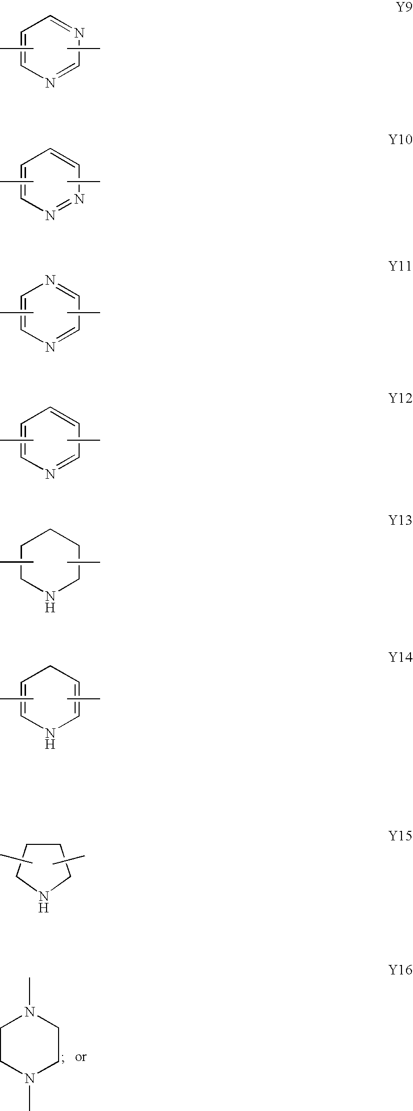
(Y12), having the two free valences in the ortho positions with respect to the nitrogen atom; (Y16) with the two valences linked to the two heteroatoms, (Y1) (pyrazol) 3,5-disubstituted; or (Y16); or
L is na=n′b=1, n′a=2, n″b=n′″b=n″a=nb=0, R₄═CH₃, R₅═R₄═R₅═H, where the precursors of the bivalent radicals X₁, as defined, or A is:
—(CO-L)ₜ-(X)ₜ₁—X₁—NO₂
where t and t1 are integers=1 and L and X are defined as above as L and X₀, but where
X₁ is a bivalent-connecting bridge is selected from the group consisting of Y—O and Y_{1,}, wherein:
for Y—O, Y is a linear or whenever possible branched C₁-C₂₀ alkylene, preferably having from 2 to 5 carbon atoms, or an optionally substituted cycloalkylene having from 5 to 7 carbon atoms; and
for Y₁, Y is selected from Y_{AR1}, Y_{AR2}, and Y_{P}, as defined above, and is more particularly: 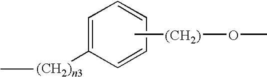
where n3 is an integer from 0 to 3; 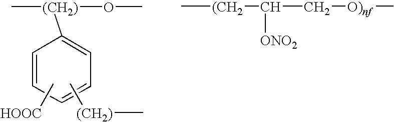
where nf′ is an integer from 1 to 6, preferably from 2 to 4; or 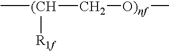
where R_{1f}═H, CH₃ and nf is an integer from 1 to 6, preferably from 2 to 4; or
A is —(CO-L)ₜ-(X)ₜ₁—X₁—NO₂
where t and t1 are integers=1 and L is defined as above;
wherein na, n′a, and n″a, equal to or different from each other, are integers from 0 to 6, preferably 1-3; nb, n′b, n″b and n′″b, are integers equal to 0 or 1; R₄ and R₅ are equal or different one from the other and are selected from the group consisting of H, linear or branched alkyls having 1 to 5 carbon atoms, preferably 1 to 3;
X is equal to O, C═O, NH, NR_{1c} where R₁ is a C₁-C₁₀, and preferably a C₁-C₄ linear or branched alkyl; OH, CH₃, Cl, N(—CH₂—CH₃)₂, SCH₂F, SH, 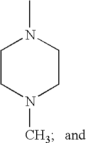
X₁ is a bivalent-connecting bridge is selected from the group consisting of Y—O and Y₁ as defined above; or
A is represented as
C(O)-L-(X₀)—(X₁)—NO₂
wherein L is (CR₄R₅)ₙₐ(O)_{nb}(CR_{4′}R_{5′})_{n′a}(CO)_{n′b}(O)_{n″b}(CO)_{n′″b}(CR_{4″}R_{5″})_{n″a}, wherein na and nb=1; R₄ and R₅═H; and wherein n′a, n″a, n′b, n″b and n′″b are 0 (thus L is CH₂O.)
X₀ is C═O, and
X₁ is the bivalent-linking group Y_{AR1}, preferably 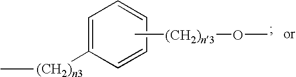
Formula B compounds being represented by: —C(O)CH₂O—X_{Z} or alternatively by —C(O)CH₂OC(O)—X_{Z} wherein X_{Z} is defined as follows: 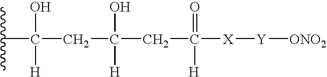
and X is O, S, NH or NHR₁, where R₁ is a straight or branched alkyl with 1 to 10 carbon atoms, preferably CH₃; and
Y is a bivalent radical having the following meanings a)-h)
a) a straight or branched C₁-C₂₀ alkylene, preferably having from 1 to 10 carbon atoms being optionally substituted with one or more of the substituents selected from the group consisting of: halogen atoms, hydroxy, —ONO₂ or T_{O}, wherein T_{O} is —OC(O)(C₁-C₁₀ alkyl)-ONO₂ or is —O(C₁-C₁₀ alkyl)-ONO₂; or alternatively, a cycloalkylene with 5 to 7 carbon atoms into cycloalkylene ring, the ring being eventually substituted with side chains T, wherein T is straight or branched alkyl with from 1 to 10 carbon atoms, preferably CH₃; 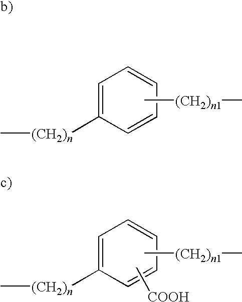
wherein for b) and c) above n is an integer from 0 to 20, and n¹ is an integer from 0 to 20; 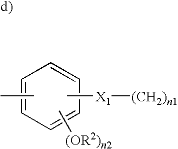
wherein, n¹ is as defined above and n² is an integer from 0 to 2; X₁ is —OCO— or —OCO— and R₂ is H or CH₃; 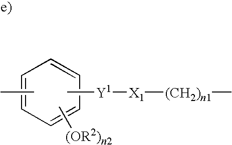
wherein n¹, n², R₂ and X₁ are as defined above; and Y¹ is either —CH₂—CH₂— or —CH₂═CH₂—(CH₂)ₙ₂—; 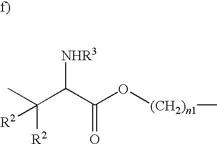
wherein n¹ and R² are as defined above; R³ is H or COCH₃; with the proviso that when Y is selected from the bivalent radicals mentioned under b) through f), the, —ONO₂ group is bound to —(CH₂)ₙ₁; 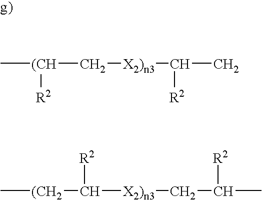
wherein X₂ is O or S, n³ is an integer from 1 to 6, preferably from 1 to 4, and R² is defined above; 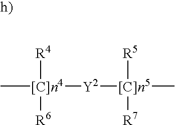
wherein: n⁴ is an integer from 0 to 10; n⁵ is an integer from 1 to 10; R⁴, R⁵, R⁶, and R⁷ are the same or different, and are H or straight or branched C₁-C₁₀ alkyl; and preferably R⁴, R⁵, R⁶, and R⁷ are H; wherein the —ONO₂ group is bound to the following structure: 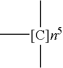
wherein, n⁵ is defined above;
Y² is a heterocyclic saturated, unsaturated or aromatic 5 or 6 members ring, containing one or more heteroatoms selected from nitrogen, oxygen, sulfur, and is selected from the following structures H1 through H13: 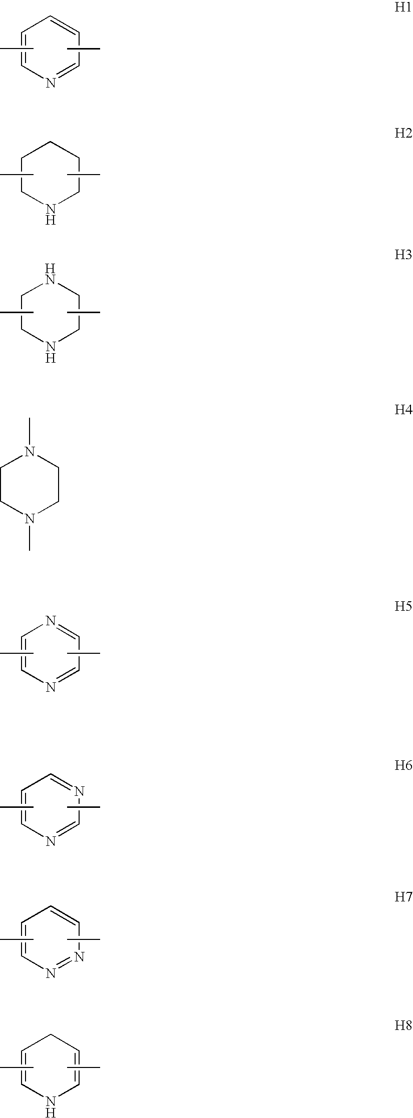 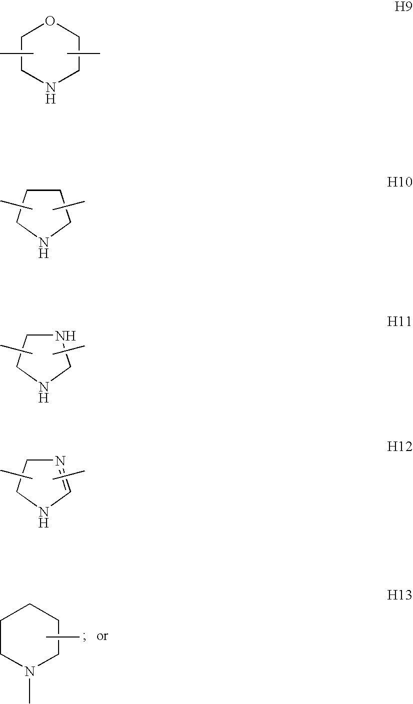
Formula C compounds being represented by: —C(O)CH₂O—(X₁—ONO₂)ₛ or alternatively by —C(O)CH₂OC(O)—(X₁—ONO₂)ₛ wherein s is an integer=1 or 2, and X₁ is a linear or when possible branched C₁-C₆ alkylene optionally substituted with at least an halogen atom, preferably having from 3 to 5 carbon atoms or X₁ is a bivalent radical equal to —(CH₂—CH₂—O)₂— or —(CH₂—CH₂—S)₂—;
Formula D compounds being represented by: —C(O)CH₂O—B—C) wherein X₁ is defined as follows: 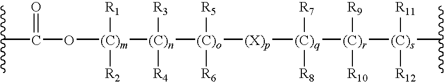
where R₁-R₁₂ are the same or different and independently are hydrogen, straight or branched C₁-C₆ alkyl, optionally substituted with aryl; m, n, o, q, r and s are each independently an integer from 0 to 6, and p is 0 or 1, and X is O, S, SO, SO₂, NR₁₃, or PR₁₃, in which R₁₃ is hydrogen, C₁-C₆ alkyl, or X is selected from the group consisting of: cycloalkylene with 5 to 7 carbon atoms into cycloalkylene ring, the ring being eventually substituted with side chains T, wherein T is straight or branched alkyl with from 1 to 10 carbon atoms, preferably CH₃; arylene, optionally substituted with one or more halogen atoms, straight or branched alkyl groups containing from 1 to 4 carbon atoms, or a straight or branched C₁-C₆ perfluoroalkyl; a 5 or 6 member saturated, unsaturated, or aromatic heterocyclic ring selected from structure H1 through H13 set forth above;
Formula E compounds being represented by: —C(O)CH₂O—B—C^{Z}, where C^{Z} is an organic nitrite or nitrate compound, or other nitric oxide donating moiety and wherein B is a spacer preferably containing 12 carbon atoms or less that connects the steroid backbone at the hydroxy immediately distal to the C-21 position with the NO donating portion of the compound, C^{Z}, via an amide, ester, carbamate or carbonate linkage that is induced adjacent to the 21 position;
wherein B—C^{Z} is R₁ wherein R₁ is selected from one of nitrite ester (—ONO), nitrate ester (—ONO₂), nitrooxyalkyls having from 1 to 20 carbons, nitrooxyalkanoyls, and nitrooxyaryls as well as but not limited to other exemplary NO donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
B—C^{Z} is equal to the following structure: 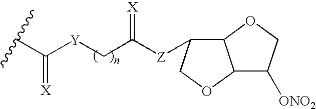
wherein n is an integer from 1 to 4; X═O or S; Y=methylene, O, or NH₂; and Z=O or NH₂; or where
W=Formula E′ where E′ is —C(O)CH₂O—(B′—C^{Z}) wherein B′ is a spacer preferably containing 12 carbon atoms or less that connects the steroid backbone at the hydroxy immediately adjacent to the C-21 position with the NO donating portion of the compound, C^{Z}, via an amide, ester, carbamate or carbonate linkage that is not adjacent to the C-21 position; or
Formula E′ wherein (B′—C^{Z}) is R′₁ wherein R′₁ is selected from nitrooxyalkyls having from 1 to 20 carbons, nitrooxyalkanoyls, and nitrooxyaryls as well as but not limited to other exemplary NO-donating moieties such as: glycerol nitrate, amylnitrate, isosorbide mononitrate, isosorbide dinitrate, mannitol nitrate, pentaerythritol nitrate, propatyl nitrate, and NO donating derivatives of the furoxans, or
R₁ can be selected from any of the following chemical moieties that have been appropriately substituted with a NO donating group: lower alkyls/alkenyls/alkynyls; that are substituted or unsubstituted; substituted or unsubstituted cyclo-alkyls/alkenyls/alkynyls; substituted or unsubstituted hetero-cycles; substituted or unsubstituted thiols, substituted or unsubstituted alkylmercaptans, nitrosothiols, and nitrosamines.
Formula F compounds being represented by: —C(O)CH₂O—K or alternatively by —C(O)CH₂OC(O)—K wherein K is defined as follows:
—Y—(CR₄R_{4′})ₚ-T-(CR₄R_{4′})ₚ—ONO₂;
—Y—(CR₄R_{4′})ₒ-[phenyl]-T-(CR₄R_{4′})ₚ—ONO₂;
wherein T is ortho, meta or para;
—Y—B-[piperazinyl]-W—(CR₄R_{4′})ₚ—ONO₂;
—Y—(CR₄R_{4′})ₚ—V—B-T-(CR₄R_{4′})ₚ—ONO₂;
—Y—(CR₄R_{4′})ₚ-T-C(O)—(CR₄R_{4′})ₚ—(CH₂)—ONO₂;
—Y—(CR₄R_{4′})ₚ—C(Z)-(CH₂)_{q}-T-(CR₄R_{4′})_{q}—(CH₂)—ONO₂;
—Y—(CR₄R_{4′})-T-(CH₂)_{q}—V—(CR₄R_{4′})_{q}—(CH₂)—ONO₂;
—Y—(CR₄R_{4′})ₚ—V—(CH₂)_{q}—V—(CR₄R_{4′})_{q}—(CH₂)—ONO₂;
—Y—(CR₄R_{4′})ₒ—(W)_{q}—(CH₂)_{q}—V—(CR₄R_{4′})ₒ—(CH₂)—ONO₂;
—NRⱼ—O—(CH₂)ₒ—V—(CR₄R_{4′})_{q}—(CH₂)—ONO₂;
—NRⱼ—O—(CH₂)ₒ—(W)_{q}—(CR₄R_{4′})_{q}—(CH₂)—ONO₂;
—O—NRⱼ—(CH₂)ₒ—(W)_{q}—(CR₄R_{4′})_{q}—(CH₂)—ONO₂;
Formula G compounds being represented by: —C(O)CH₂O—X1 or alternatively by —C(O)CH₂OC(O)—X1 wherein X1 is defined as follows:
—C=A-R₂ wherein A is (CH), N, or S and wherein R₂ is a lone pair of electrons, a nitrile group, a nitro group, an alkylsulfonyl group, an arylsulfonyl group, an alkylcarbonyl group, a carboxamido group, a carboxylic ester or a cycloalkylalkyl group;
or alternatively, X1 is equal to K which is further defined as:
—Wₐ-E_{b}-[C—(Rₑ)(R_{f})]ₚ-E_{c}-[C—(Rₑ)(R_{f})]ₓ—W_{d}—[C—(Rₑ)(R_{f})]_{y}—(U)—(V);
where, —(U)—(V) is equal to —Wᵢ-Eⱼ-W_{g}-[C—(Rₑ)(R_{f})]_{z}-T-Q; or alternatively
U and V are taken independently wherein U is O, S, or —N(Rₐ)(Rᵢ) and V is nitro, nitroso, or hydrogen;
wherein, a, b, c, d, g, i and j are each independently an integer from 0 to 3;
p, x, y and z are each independently an integer from 0 to 10;
Wₐ, W_{d}, Wᵢ, and W_{g} are independently —C(O)—, —C(S)—, -T-, —[C—(Rₑ)(R_{f})]ₕ—, an alkyl group, an aryl group, a heterocyclic ring, an arylheterocyclic ring, or —(CH₂CH₂O)_{q}—;
E at each occurrence is independently -T-, an alkyl group, an aryl group, —[C—(Rₑ)(R_{f})]ₕ—, a heterocyclic ring, an arylheterocyclic ring, or —(CH₂CH₂O)_{q}—;
h is an integer form 1 to 10;
q is an integer of from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, a cycloalkylalkyl, a heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, an alkylsulfonic acid, an arylsulfonic acid, an arylalkoxy, an alkylthio, an arylthio, a cyano, an aminoalkyl, an aminoaryl, an alkoxy, an aryl, an arylalkyl, an alkylaryl, a carboxamido, a alkyl carboxamido, an aryl carboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, a sulfonic ester, a carbamoyl, a urea, a nitro, -T-Q, or —[C—(Rₑ)(R_{f})]ₖ-T-Q; or Rₑ and R_{f} taken together with the carbon atom to which they are attached are a carbonyl, a methanthial, a heterocyclic ring, a cycloalkyl group or a bridged cycloalkyl group;
k is an integer from 1 to 3;
T at each occurrence is independently a covalent bond, a carbonyl, an oxygen, —S(O)ₒ— or —N(Rₐ)(Rᵢ)—;
o is an integer from 0 to 2;
Rₐ is a lone pair of electrons, a hydrogen or an alkyl group;
Rᵢ is a hydrogen, an alkyl, an aryl, an alkylcarboxylic acid, an aryl carboxylic acid, an alkylcarboxylic ester, an arylcarboxylic ester, an alkylcarboxamido, an arylcarboxamido, an alkylaryl, an alkylsulfinyl, an alkylsulfonyl, an arylsulfinyl, an arylsulfonyl, a sulfonamido, a carboxamido, a carboxylic ester, an amino alkyl, an amino aryl, —CH₂—C-(T-Q)(Rₑ)(R_{f}) or —(N₂O₂)⁻.M⁺ wherein M⁺ is an organic or inorganic cation, with the proviso that when Rᵢ is —CH₂—C-(T-Q)(Rₑ)(R_{f}) or —(N₂O₂)⁻.M⁺ or when Rₑ or R_{f} are T-Q or —[C—(Rₑ)(R_{f})]ₖ-T-Q, then the “-T-Q” subgroup designated as X can be a hydrogen, an alkyl, an alkoxy, an alkoxyalkyl, an aminoalkyl, a hydroxy, a heterocyclic ring or an aryl group, wherein, in cases where Rₑ and R_{f} are a heterocyclic ring or taken together Rₑ and R_{f} are a heterocyclic ring, then Rᵢ can be a substituent on any disubstituted nitrogen contained within the radical where Rᵢ is as defined herein.

45. The method of claim 41, wherein the compound has the structure of Formula 1: 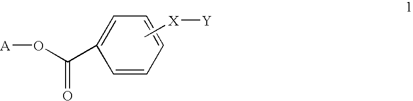
wherein A is a steroid residue;
X is a C₁-C₅ branched or linear chain alkyl; and
Y is either (ONO₂) or (ONO); with the proviso that A is linked to the remainder of Formula 1 by way of the C-11 or the C-17 position.

46. The method of claim 45, wherein the steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

47. The method of claim 45, wherein A is budesonide, X is CH₂ and Y is (ONO₂) and A is linked to the remainder of Formula 1 through the C-17 position.

48. The method of claim 45, wherein the steroid residue is a corticosteroid budesonide and the NO-donor is linked to budesonide to provide a compound of Formula 2:

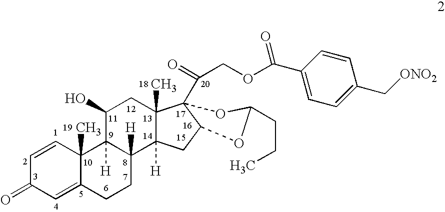

49. The method of claim 41, wherein the respiratory disease is selected from the group consisting of asthma, chronic obstructive pulmonary disorder, eosinophilic cough, bronchitis, acute and chronic rejection of lung allograft, sarcoidosis, pulmonary fibrosis, rhinitis, pulmonary hypertension, cystic fibrosis, bronchiectasis and sinusitis.

50. The use according to claim 49, wherein the respiratory disease is asthma.

51. The method of claim 49, wherein the respiratory disease is chronic obstructive pulmonary disorder.

52. The method of claim 41, wherein the compound is administered via inhalation at a dose from about 100 μg to about 3000 μg.

53. The method of claim 52, wherein the compound is administered via inhalation at a dose from about 200 μg to about 2400 μg.

54. The method of claim 52, wherein the compound is administered via inhalation at a dose from about 400 μg to about 1500 μg.

55. The method of claim 52, wherein the compound is administered via inhalation at a dose from about 600 μg to about 1200 μg.

56. The method of claim 52, wherein the compound is administered via inhalation at a dose from about 800 μg to about 1200 μg.

57. The method of claim 41, wherein the compound is administered twice daily.

58. The method of claim 41, wherein the compound is administered as needed.

59. The method of claim 41, wherein the compound is administered orally at a dose from about 100 μg to about 250 mg.

60. The method of claim 41, wherein the compound is administered intravenously at a dose from about 100 μg to about 1 g.

61. The method of claim 1 wherein the medicament also provides a statistically significant decrease in sputum neutrophil levels.

62. The method of claim 41 wherein the medicament also provides a statistically significant decrease in sputum neutrophil levels.

63. The method of claim 1 wherein the medicament also provides a statistically significant decrease in plasma CRP level in patients having a respiratory disease.

64. The method of claim 21 wherein the medicament also provides a statistically significant decrease in plasma CRP level in patients having a respiratory disease.

65. The method of claim 1 wherein the medicament also provides a statistically significant decrease in sputum neutrophil levels and a statistically significant decrease in plasma CRP level in patients having a respiratory disease

65. The method of claim 1, wherein the sputum neutrophil level of the patients is greater than 20%.

66. The method of claim 1, wherein W is Formula A.

67. The method of claim 1, wherein W is Formula B.

68. The method of claim 1, wherein W is Formula C.

69. The method of claim 1, wherein W is Formula D.

70. The method of claim 1, wherein W is Formula E.

71. The method of claim 1, wherein W is Formula F.

72. The method of claim 1, wherein W is Formula G.

73. A method for treating a respiratory disease in patients without affecting free urinary cortisol levels compared to pre-treatment levels in the patients, said compound having Formula 3:
A-W 3
wherein A is a steroid residue, and
W is any nitric oxide (“NO”) donating moiety linked to the steroid backbone and is capable of donating, releasing and/or directly or indirectly transferring any of the three redox forms of nitrogen monoxide (NO⁺, NO⁻, NO.).

74. The method of claim 1 such that the free urinary cortisol levels remain unchanged from the pre-treatment levels in the patients.

75. The method of claim 73 wherein steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

76. The method of claim 73, wherein the compound has the structure of Formula 1: 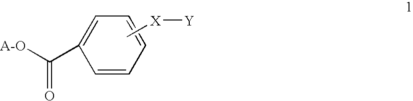
wherein A is a steroid residue;
X is a C₁-C₅ branched or linear chain alkyl; and
Y is either (ONO₂) or (ONO); with the proviso that A is linked to the remainder of Formula 1 by way of the C-11, C-17, or C-21 position.

79. The method of claim 73, wherein the steroid residue is a corticosteroid budesonide and the NO-donor is linked to budesonide to provide a compound of Formula 2:

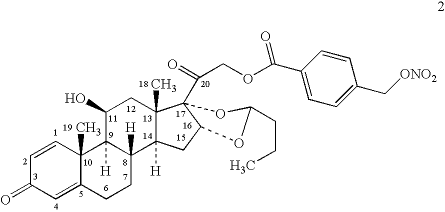

80. A method for treating respiratory disease in patients such that there is a statistically significant decrease in the patients of a condition selected from sputum neutrophil levels, blood pressure, and CRP levels, said compound having Formula 3:
A-W 3
wherein A is a steroid residue, and
W is any nitric oxide (“NO”) donating moiety linked to the steroid backbone and is capable of donating, releasing and/or directly or indirectly transferring any of the three redox forms of nitrogen monoxide (NO⁺, NO⁻, NO.).

81. The method of claim 80 wherein steroid residue A is selected from the group consisting of 21-acetoxypregnenolone, alclometasone, algestone, amcinonide, beclomethasone, beclomethasone dipropionate, betamethasone, budesonide, chlorprednisone, ciclesonide, clobetasol, clocortolone, cloprednol, corticazol, corticosterone, cortisone, deflazacort, desonide, desoxicorticosterone, dexamethasone, diflorasone, diflucortolone, difluprednate, enoxolone, fluazacort, fluocinolone acetonide, flucloronide, flumethasone, flunisolide, fluorometholone, fluocinonide, fluocortin-butyl, fluocortolone, fluperolone acetate, fluprednidene acetate, fluprednisolone, flurandrenolide, fluticasone, fluticasone propionate, fluticasone fluoronate, formocortal, halcinonide, halometasone, haloprednone acetate, hydrocortamate, hydrocortisone, hydrocortisone phosphate, hydrocortisone terbutate, mazipredone, medrysone, meprednisone, methylprednisolone, mometasone furoate, paramethasone, prednicarbate, prednisone, prednisolone 21-diethylaminoacetate, prednisolone sodium succinate, prednisolone sodium phosphate, prednisplone sodium 21-m-sulfo-benzoate, prednisolone 21-stearoylglycolate, prednisolone terbutate, prednisolone 21-trimethylacetate, prednival, prednylidene, prednylidene 21-diethylaminoacetate, tixocortol, triamcinolone benetonide, triamcinolone hexacetonide, and triamcinolone acetonide.

82. The method of claim 80-81, wherein the compound has the structure of Formula 1: 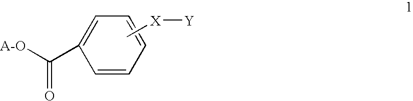
wherein A is a steroid residue;
X is a C₁-C₅ branched or linear chain alkyl; and
Y is either (ONO₂) or (ONO); with the proviso that A is linked to the remainder of Formula 1 by way of the C-11, C-17, or C-21 position.

83. The method of claim 80, wherein the steroid residue is a corticosteroid budesonide and the NO-donor is linked to budesonide to provide a compound of Formula 2: [chemical structure]
